SEQUENCE LISTING (5 sequences)

SEQ ID NO: 1 - DNA, 14 bp, Homo sapiens
acaggggtgtgggg

SEQ ID NO: 2 - DNA, 18 bp, Artificial
  synthetic oligonucleotide
ccctaaccctaaccctaa

SEQ ID NO: 3 - DNA, 24 bp, Artificial
  synthetic oligonucleotide
ccctaaccctaaccctaaccctaa

SEQ ID NO: 4 - DNA, 54 bp, Artificial
  synthetic oligonucleotide
ccctaaccctaaccctaaccctaatttttagtcaaagcatgaagttaccgtttt

SEQ ID NO: 5 - DNA, 45 bp, Artificial
  synthetic oligonucleotide
ccctaaccctaaccctaaccctaatttttctcttggaaagaaagt

(19) United States
(12) Patent Application Publication
(10) Pub. No.: US 2011/0195864 A1
(43) Pub. Date: Aug. 11, 2011

(54) Assays for determining telomere length and repeated sequence copy number

(75) Inventor: Yunqing Ma, San Jose, CA (US)

(73) Assignee: Affymetrix, Inc., Santa Clara, CA (US)

(21) Appl. No.: 13/066,212

(22) Filed: Apr. 8, 2011

Related U.S. Application Data
(62) Division of application No. 12/454,746, filed on May 21, 2009.
(60) Provisional application No. 61/130,266, filed on May 28, 2008.

(51) Int. Cl. C40B 30/04; C12Q 1/68

(52) U.S. Cl. 506/9; 435/6.11

(57) ABSTRACT

Methods of detecting copy number of a repeated sequence element, including methods of determining telomere length, are provided. The methods can be multiplexed for detection of repeated sequence element copy number on two or more nucleic acid targets simultaneously. Compositions, kits, and systems related to the methods are also described.

### CROSS-REFERENCE TO RELATED APPLICATIONS

This application is a non-provisional utility patent application claiming priority to and benefit of the following prior provisional patent application: U.S. Ser. No. 61/130,266, filed May 28, 2008, entitled “ASSAYS FOR DETERMINING TELOMERE LENGTH AND REPEATED SEQUENCE COPY NUMBER” by Yunqing Ma, which is incorporated herein by reference in its entirety for all purposes.

### FIELD OF THE INVENTION

The present invention is in the field of nucleic acid analysis. The invention includes methods for determining copy number of tandemly repeated sequence elements, including methods for determining telomere length. The invention also includes compositions and kits related to the methods.

### BACKGROUND OF THE INVENTION

Telomeres, regions of repetitive DNA at the ends of eukaryotic chromosomes, play a key role in the maintenance of chromosomal stability. For example, telomeres can protect chromosomes from shortening during replication with each cell division, recombination, fusion to other chromosomes, and degradation by nucleases.

Telomeres include a number of noncoding tandem DNA repeats. In vertebrates and some other eukaryotes, the telomeric repeat is the hexanucleotide repeat TTAGGG (or equivalently its complement on the opposite strand of the chromosome, CCCTAA). Telomeric repeat sequences have also been determined for a variety of other organisms, including, for example, Tetrahymena (TTGGG), Oxytricha (TTTTGGGG), Arabidopsis thaliana and many other plants (TTTAGGG), Chlamydomonas (TTTTAGGG), and many yeasts.

Telomere length varies widely among species, e.g., from an average of 300-600 bp in yeast to 20 kb or more in higher eukaryotes. Telomere length also varies within species, where it can be affected by factors such as an individual organism's age, stress level, or disease state (e.g., Steinert et al. (2002) “Telomere biology and cellular aging in nonhuman primate cells” Exper Cell Res 272:146-152, Epel et al. (2004) “Accelerated telomere shortening in response to life stress” Proc Natl Acad Sci 101:17312-17315, and Richards et al. (2007) “Higher serum vitamin D concentrations are associated with longer leukocyte telomere length in women” Am J Clin Nutr 86:1420-1425). For instance, leukocyte telomere dynamics are ostensibly a biological indicator of human aging, and estimation of human age based on telomere shortening has been suggested in forensics (Tsuji et al. (2002) “Estimating age of humans based on telomere shortening” Forensic Sci Int. 126(3):197-9).

Telomere length maintenance has been implicated in a number of human diseases. For example, measurement of telomere length can diagnose dyskeratosis congenita, a form of aplastic anemia, where patients' peripheral blood white cells have very short telomeres (Alter et al. (2007) “Very short telomere length by flow fluorescence in situ hybridization identifies patients with dyskeratosis congenita” Blood 110(5):1439-47). Telomere length is also of considerable interest in a number of human cancers. For example, studies have shown that short telomere length is associated with increased risk for human cancers such as bladder, head and neck, lung, and renal cell cancer (Shen et al. (2007) “Short Telomere Length and Breast Cancer Risk: A Study in Sister Sets” Cancer Research 67:5538-5544, Suleman (2003) “Telomere length analysis as a novel diagnostic test for bladder cancer” Enquiries J Interdisciplinary Studies for High School Students 1:1-5, and Zhou et al. (2005) “Telomere length of transferred lymphocytes correlates with in vivo persistence and tumor regression in melanoma patients receiving cell transfer therapy” J Immunol 175:7046-7052, and Chin et al. (2004) “In situ analyses of genome instability in breast cancer” Nat Genet. 36(9):984-8). Telomere length in human white blood cells is shorter in breast cancer patients (Levy et al. (1998) “Telomere length in human white blood cells remains constant with age and is shorter in breast cancer patients” Anticancer Res. 18(3A):1345-9), and telomere length in breast cancer patients can be remarkably changed before and after chemotherapy with or without stem cell transplantation (Schroder et al. (2001) “Telomere length in breast cancer patients before and after chemotherapy with or without stem cell transplantation” British Journal of Cancer 84:1348-1353). It has been shown that total telomere length is shorter in invasive breast cancer than in normal breast tissue. In addition, a recent study suggested that an increased level of telomere shortening on 17q may be involved in chromosome instability and the progression of duct carcinoma in situ (DCIS; Fariborz et al. (2007) “Telomere length on chromosome 17q shortens more than global telomere length in the development of breast cancer” Neoplasia 9:265-270).

Information on telomere length variation, in total or on individual chromosome(s), is therefore valuable for diagnosis, prognosis, and treatment of many conditions. For example, the profile of telomere length for a given tumor could help predict prognosis and guide choice of most appropriate treatment. Convenient methods for measurement of telomere length are also desirable for other applications, for example, in forensic science and in the development of telomerase-inhibiting drugs (for a review of telomerase as an anti-cancer target, see Harley (2008) “Telomerase and cancer therapeutics” Nature Reviews 8:1-14).

Current techniques for measurement of telomere length include quantitative PCR, Southern blot analysis, quantitative fluorescence microscopy (Q-FISH), and flow cytometry (flow-FISH); see, e.g., Shen et al. supra, Baerlocher et al. (2002) “Telomere length measurement by fluorescence in situ hybridization and flow cytometry: Tips and pitfalls” Cytometry 47:89-99, Cawthon (2002) “Telomere measurement by quantitative PCT” Nuc. Acids Res. 30:e47, Chiang et al. (2006) “Generation and characterization of telomere length maintenance in tankyrase 2-deficient mice” Mol Cell Biol 26:2037-2043, Baird et al. (2004) “Normal telomere erosion rates at the single cell level in Werner syndrome fibroblast cells” Hum Mol Genet 13:1515-1524, Britt-Compton et al. (2006) “Structural stability and chromosome-specific telomere length is governed by cis-acting determinants in humans” Hum Mol Genet 15:725-733, Liu et al. (2002) “Preferential maintenance of critically short telomeres in mammalian cells heterozygous for mTert” Proc Natl Acad Sci 99:3597-3602, and de Deken et al. (1998) “Decrease of telomere length in thyroid adenomas without telomerase activity” Journal of Clinical Endocrinology and Metabolism 83:4368-4372. However, these methods generally have one or more of the following drawbacks: requires purification of DNA, is time consuming, has poor precision, requires high sample input (e.g., 1.5-2 million cells for FISH method), or has low sensitivity. In particular, none of the current techniques can easily measure the telomere length of individual chromosomes.

Among other aspects, the present invention provides methods that overcome the above noted limitations and permit rapid, simple, and sensitive detection of telomere length as either an average over multiple chromosomes or for one or more single chromosomes. In addition, the methods also facilitate analysis of other tandem repeated sequence elements. A complete understanding of the invention will be obtained upon review of the following.

### SUMMARY OF THE INVENTION

Methods of determining copy number of a repeated sequence element, including methods of determining telomere length, are provided herein. The methods are optionally multiplexed for detection of repeated sequence element copy number on two or more nucleic acid targets simultaneously. Compositions, kits, and systems related to the methods are also described.

Accordingly, a first general class of embodiments provides methods of detecting copy number of a repeated sequence element that is present in multiple tandem copies on a first nucleic acid target molecule. In the methods, a test sample comprising the first nucleic acid target molecule is provided. Multiple copies of a label extender are provided. Each copy of the label extender is capable of hybridizing to at least one copy of the repeated sequence element or to a subsequence thereof. A label probe system comprising a label, wherein a component of the label probe system is capable of hybridizing to the label extender, is also provided.

The label extender copies and the copies of the repeated sequence element or subsequence thereof on the first nucleic acid target molecule are hybridized, and the label probe system is hybridized to the label extender copies. A signal from the label is detected, and its intensity is correlated with a number of copies of the repeated sequence element and/or with a length of the first nucleic acid target molecule occupied by the copies of the repeated sequence element.

In one aspect, the first nucleic acid target molecule is captured on a solid support prior to detecting the signal from the label. In one class of embodiments, the first nucleic acid target molecule is captured on the solid support by providing a first set of one or more capture extenders, which first set of capture extenders is capable of hybridizing to the first nucleic acid target molecule, hybridizing the first set of capture extenders to the first nucleic acid target molecule, and associating the first set of capture extenders with the solid support, whereby hybridizing the first set of capture extenders to the first nucleic acid target molecule and associating the first set of capture extenders with the solid support captures the first nucleic acid target molecule on the solid support. Optionally, a first capture probe is bound to the solid support, and the first set of capture extenders is associated with the solid support by hybridizing the capture extenders to the first capture probe. In some embodiments, the first set of capture extenders comprises a single capture extender that is capable of hybridizing to at least one copy of the repeated sequence element or to a subsequence thereof. Multiple copies of the single capture extender are provided. In other embodiments, the one or more capture extenders of the first set hybridize to one or more polynucleotide sequences in the first nucleic acid target molecule other than the repeated sequence element or a subsequence thereof.

In one exemplary class of embodiments, the label probe system comprises a preamplifier, a plurality of amplification multimers, and a multiplicity of label probes, wherein the preamplifier is capable of hybridizing simultaneously to the label extender and to the plurality of amplification multimers, and wherein the amplification multimer is capable of hybridizing simultaneously to the preamplifier and to a plurality of the label probes. In other exemplary embodiments, the label probe system includes an amplification multimer and a plurality of label probes. In one class of embodiments, the label probe comprises the label; in other embodiments, the label probe is configured to bind a label.

The first nucleic acid target molecule can be essentially any desired nucleic acid, including but not limited to, DNA, RNA, eukaryotic, bacterial and/or viral genomic RNA and/or DNA (double-stranded or single-stranded), and extra-genomic DNA. In one class of embodiments, the first nucleic acid target molecule comprises a chromosome or portion thereof. The first nucleic acid target molecule can comprise a distal portion of a chromosome arm and the repeated sequence element can be a telomeric repeat, e.g., in embodiments in which telomere length is to be analyzed.

Exemplary repeated sequence elements of particular interest in the context of the present invention include, but are not limited to, telomeric repeats, short tandem repeats, variable number of tandem repeats, microsatellite repeats, minisatellite repeats, and trinucleotide repeats, as well as other tandemly repeated elements where multiple (at least two, e.g., 3, 4, or 5 or more) repeats are immediately adjacent to each other. Typically, the repeated sequence element is present in at least 10 tandem copies on the first nucleic acid target molecule, and more typically in at least 20 tandem copies, at least 30 tandem copies, at least 40 tandem copies, at least 50 tandem copies, or at least 100 tandem copies. Optionally, the repeated sequence element is present in at least 250, at least 500, at least 1000, at least 2000, or even at least 3000 tandem copies on the first nucleic acid target molecule.

The repeated sequence element to be analyzed can be essentially any desired repeated element of any length (e.g., 500 nucleotides or less, 250 nucleotides or less, 200 nucleotides or less, 150 nucleotides or less, or 100 nucleotides or less in length). More typically, however, each copy of the repeated sequence element is 50 nucleotides or less in length, for example, 25 nucleotides or less, 24 nucleotides or less, 22 nucleotides or less, 20 nucleotides or less, 15 nucleotides or less, or even 10 nucleotides or less in length.

Depending, e.g., on the length of the repeated sequence element, the label extender can hybridize to a subsequence of the element (e.g., for longer elements), to the entirety of a single copy of the element, or to at least two tandem copies of the repeated sequence element (e.g., for shorter elements). Optionally, the label extender is capable of hybridizing to at least three, four, five, or more tandem copies of the repeated sequence element.

The methods can be conveniently multiplexed to analyze the repeated sequence element on two or more nucleic acid molecules simultaneously. Thus, in one class of embodiments, the test sample also comprises a second nucleic acid target molecule that is distinct from the first nucleic acid target molecule and that comprises multiple tandem copies of the repeated sequence element. The methods include hybridizing the label extender copies to the copies of the repeated sequence element or subsequence thereof on the second nucleic acid target molecule. The label probe system is hybridized to the label extenders and signal is detected as described above. Third, fourth, fifth, etc. (or even tenth, twentieth, fiftieth, hundredth, etc.) nucleic acid target molecules comprising the repeated sequence element are optionally included in the test sample and detected with the label extender as noted for the second target.

The first and second (and optional third, fourth, etc.) nucleic acid target molecules are optionally captured on a solid support. If an average repeated sequence element copy number or length occupied by the element is desired for the target molecules, then the molecules can be captured in a single well of a multi well plate, on a single spot on an array, on a single set of particles, or the like. If the copy number or length occupied by the repeated sequence element on each separate molecule is desired, however, then different target molecules are conveniently captured at different positions in an array, on different distinguishable sets of particles, or the like.

Thus, in one class of embodiments, the solid support is a substantially planar solid support, and the first nucleic acid target molecule is captured at a first selected position on the solid support and the second nucleic acid target molecule is captured at a second selected position on the solid support. The signal from the label is then detected at each different selected position on the solid support. The intensity of the signal for a given position is correlated with the number of copies of the repeated sequence element on the corresponding nucleic acid target molecule and/or with the length of the corresponding nucleic acid target molecule occupied by the copies of the repeated sequence element.

In a related class of embodiments, the solid support comprises a population of particles that includes at least two sets of particles, the particles in each set being distinguishable from the particles in every other set. The first nucleic acid target molecule is captured on a first set of the particles, and the second nucleic acid target molecule is captured on a second set of the particles. At least a portion of the particles from each set is identified, and the signal from the label on those particles is detected. The intensity of the signal for a given set of particles is correlated with the number of copies of the repeated sequence element on the corresponding nucleic acid target molecule and/or with the length of the corresponding nucleic acid target molecule occupied by the copies of the repeated sequence element.

The first, second, third, etc. nucleic acid targets are optionally captured as described for single targets above, e.g., using capture extenders and capture probes. Thus, in one exemplary class of embodiments, capturing the first nucleic acid target molecule on a solid support comprises providing a first set of one or more capture extenders, which first set of capture extenders is capable of hybridizing to the first nucleic acid target molecule, hybridizing the first set of capture extenders to the first nucleic acid target molecule, and associating the first set of capture extenders with the solid support, whereby hybridizing the first set of capture extenders to the first nucleic acid target molecule and associating the first set of capture extenders with the solid support captures the first nucleic acid target molecule on the solid support, and capturing the second nucleic acid target molecule on a solid support comprises providing a second set of one or more capture extenders, which second set of capture extenders is capable of hybridizing to the second nucleic acid target molecule, hybridizing the second set of capture extenders to the second nucleic acid target molecule, and associating the second set of capture extenders with the solid support, whereby hybridizing the second set of capture extenders to the second nucleic acid target molecule and associating the second set of capture extenders with the solid support captures the second nucleic acid target molecule on the solid support. The second set of capture extenders can be identical to or distinct from the first set of capture extenders. For example, the first and second sets of capture extenders can be identical where an average copy number or length occupied by the element is desired for the first and second target molecules (e.g., a single capture extender complementary to at least one copy of the repeated sequence element or a subsequence thereof or a set of capture extenders complementary to a sequence present on both targets can be employed). Where the different targets are to be captured to different sets of particles or different positions on an array, however, distinct sets of capture extenders are generally employed for the different targets, e.g., complementary to sequences unique to each particular target.

The copy number of and/or length occupied by the repeated sequence element obtained by the methods is optionally expressed in relative or absolute terms. If desired, the copy number or length can be expressed per target molecule, per chromosome, per cell, per μg of nucleic acid, or the like, e.g., by normalization with respect to a reference nucleic acid. Accordingly, in one class of embodiments a standard function for cell number or amount of cellular nucleic acid input versus quantity of a reference nucleic acid is provided. The reference nucleic acid is quantitated from the test sample. A cell number or amount of cellular nucleic acid is determined for the test sample based on the standard function and the quantity of reference nucleic acid in the test sample, and the intensity of the signal, the number of copies, and/or the length is normalized to the cell number or amount of cellular nucleic acid. Exemplary reference nucleic acids include, but are not limited to, a ribosomal DNA (e.g., an 18S rDNA, 5.8S rDNA, or 28S rDNA), an Alu sequence, and a β-globin gene.

A related general class of embodiments provides methods of determining telomere length by detecting telomeric repeats present on a first chromosome arm. In the methods, a sample comprising the first chromosome arm or a distal portion thereof is provided. Multiple copies of a label extender that is capable of hybridizing to at least one copy of the telomeric repeat are provided. A label probe system comprising a label, wherein a component of the label probe system is capable of hybridizing to the label extender, is also provided. The label extender copies are hybridized to the telomeric repeats on the first chromosome arm or portion thereof, and the label probe system is hybridized to the label extender copies. A signal from the label is detected, and its intensity is correlated with a number of copies of the telomeric repeat and/or with the length of the telomere.

The first chromosome arm or distal portion thereof is optionally captured to a solid support prior to detecting the signal from the label. Such capture can involve, e.g., hybridization to capture extenders and a capture probe as described for the methods above. Exemplary suitable supports are described herein.

The label extender optionally hybridizes to two or more tandem copies of the telomeric repeat. For example, the label extender can hybridize to at least three, four, five, or more tandem copies of the telomeric repeat.

The methods of determining telomere length are conveniently employed to determine average telomere length over two or more chromosomes or arms or multiplexed to determine telomere length of two or more chromosomes or arms simultaneously in a single assay. Thus, in one aspect, the sample comprises a second chromosome arm or a distal portion thereof, and the methods include hybridizing the label extender copies to the telomeric repeats on the second chromosome arm or portion thereof. To determine average telomere length, the intensity of the signal is correlated with an average of the number of copies of the telomeric repeat present on the first and second chromosome arms and/or with an average of the length of the telomere on the first and second chromosome arms. For determination of individual telomere lengths, the first and second chromosome arms or distal portions thereof are captured to different selected positions on a solid support or to different distinguishable sets of particles prior to detecting the signal from the label, and the intensity measured for a selected position on the solid support or for a selected set of particles is correlated with the number of copies of the telomeric repeat present on the corresponding chromosome arm and/or with the length of the corresponding chromosome arm.

The methods are readily applied to more than two arms. Thus, more generally, in one class of embodiments the sample is derived from an organism having n chromosomes in its haploid genome, the sample comprises 2n chromosome arms or distal portions thereof, and the methods include hybridizing the label extender to at least one telomeric repeat on each chromosome arm or portion thereof. For determination of average telomere length, the intensity of the signal is correlated with an average of the number of copies of the telomeric repeat present on the 2n chromosome arms and/or with an average of the length of the telomere on the 2n chromosome arms. For determination of individual telomere lengths, the 2n chromosome arms or distal portions thereof are captured to different selected positions on a solid support or to different distinguishable sets of particles prior to detecting the signal from the label, and the intensity of the signal measured for a selected position on the solid support or for a selected set of particles is correlated with the number of copies of the telomeric repeat present on the corresponding chromosome arm and/or with the length of the corresponding chromosome arm. Again, capture of the various chromosome arms can involve, e.g., hybridization to capture extenders and a capture probe or probes as described for the methods above.

Essentially all of the features noted for the methods above apply to these embodiments as well, as relevant; for example, with respect to composition of the label probe system (e.g., inclusion of preamplifier, amplification multimer, and/or label probe), type of label, inclusion of blocking probes, source of the nucleic acid and/or test sample, type of solid support, use of a reference nucleic acid for normalization, and/or the like. As for the embodiments described above, the number of copies of the telomeric repeat or telomere length is optionally expressed in relative or absolute terms.

As noted, compositions related to the methods are also a feature of the invention. Thus, one general class of embodiments provides a composition that includes a first set of one or more capture extenders, which first set of capture extenders is capable of hybridizing to a first nucleic acid target molecule that comprises multiple tandem copies of a repeated sequence element; a label extender, which label extender is capable of hybridizing to at least one copy of the repeated sequence element or to a subsequence thereof; and a label probe system comprising a label, wherein a component of the label probe system is capable of hybridizing to the label extender. The composition optionally includes the first nucleic acid target molecule (e.g., in a test sample).

Essentially all of the features noted for the methods above apply to these embodiments as well, as relevant; for example, with respect to composition of the label probe system, type of label, type, length, and/or copy number of the repeated sequence element, source of the nucleic acid and/or test sample, configuration of the label extender, inclusion of blocking probes, a second (third, fourth, etc.) set of capture extenders for a second (third, fourth, etc.) nucleic acid target molecule, the second (third, fourth, etc.) target nucleic acid molecule, a solid support, capture probe(s), a reference nucleic acid, a set of one or more capture extenders capable of hybridizing to the reference nucleic acid, and/or at least one label extender capable of hybridizing to the reference nucleic acid, and/or the like.

Yet another general class of embodiments provides a kit for determining copy number of a repeated sequence element present in multiple tandem copies on a first nucleic acid target molecule. The kit includes a first set of one or more capture extenders, which first set of capture extenders is capable of hybridizing to the first nucleic acid target molecule; a label extender, which label extender is capable of hybridizing to at least one copy of the repeated sequence element or to a subsequence thereof; and a label probe system comprising a label, wherein a component of the label probe system is capable of hybridizing to the label extender; packaged in one or more containers.

The kit optionally also includes instructions for using the kit, one or more buffered solutions, one or more standards comprising one or more nucleic acids at known concentration, a second (third, fourth, etc.) set of one or more capture extenders for a second (third, fourth, etc.) nucleic acid target molecule, blocking probes, a solid support (e.g., a spatially addressable support or population of sets of identifiable particles), capture probe(s) (e.g., a single capture probe on a solid support, or an array of capture probes on a spatially addressable solid support or on distinguishable sets of particles), a set of one or more capture extenders capable of hybridizing to a reference nucleic acid, and/or at least one label extender capable of hybridizing to the reference nucleic acid.

Essentially all of the features noted for the methods above apply to these embodiments as well, as relevant; for example, with respect to composition of the label probe system, type of label, type, length, and/or copy number of the repeated sequence element, source of the nucleic acid and/or test sample, configuration of the label extender, and/or the like.

### BRIEF DESCRIPTION OF THE DRAWINGS

FIG. 1 schematically illustrates a typical standard bDNA assay.

FIG. 2 Panels A-E schematically depict a multiplex assay in which different nucleic acid targets are captured on different distinguishable subsets of microspheres, a label extender that recognizes the repeated sequence element is hybridized to the target nucleic acid molecules, microspheres from the different subsets are identified, and signal from a label probe captured on those microspheres is detected.

FIG. 3 Panels A-D schematically depict a multiplex assay in which different nucleic acid targets are captured at different selected positions on a solid support. Panel A shows a top view of the solid support, while Panels B-D show the support in cross-section.

FIG. 4 Panels A-B schematically depict an assay in which a mixture of different nucleic acid targets are captured together on a solid support, for determining an average of the repeated sequence elements present on the targets rather than a value for each individual target. The support is shown in cross-section.

FIG. 5 presents a graph illustrating determination of average telomere length.

Schematic figures are not necessarily to scale.

### DEFINITIONS

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present invention, the preferred materials and methods are described herein. Accordingly, the terminology used herein is for the purpose of describing particular embodiments only, and is not intended to be limiting.

As used in this specification and the appended claims, the singular forms “a,” “an” and “the” include plural referents unless the context clearly dictates otherwise. Thus, for example, reference to “a molecule” includes a plurality of such molecules, and the like.

The term “about” as used herein indicates the value of a given quantity varies by +/−10% of the value, or optionally +/−5% of the value, or in some embodiments, by +/−1% of the value so described.

The term “polynucleotide” (and the equivalent term “nucleic acid”) encompasses any physical string of monomer units that can be corresponded to a string of nucleotides, including a polymer of nucleotides (e.g., a typical DNA or RNA polymer), peptide nucleic acids (PNAs), modified oligonucleotides (e.g., oligonucleotides comprising nucleotides that are not typical to biological RNA or DNA, such as 2′-O-methylated oligonucleotides), and the like. The nucleotides of the polynucleotide can be deoxyribonucleotides, ribonucleotides or nucleotide analogs, can be natural or non-natural (e.g., Locked NucleicAcid™, isoG, or isoC nucleotides), and can be unsubstituted, unmodified, substituted or modified. The nucleotides can be linked by phosphodiester bonds, or by phosphorothioate linkages, methylphosphonate linkages, boranophosphate linkages, or the like. The polynucleotide can additionally comprise non-nucleotide elements such as labels, quenchers, blocking groups, or the like. The polynucleotide can be, e.g., single-stranded or double-stranded.

A “polynucleotide sequence” or “nucleotide sequence” is a polymer of nucleotides (an oligonucleotide, a DNA, a nucleic acid, etc.) or a character string representing a nucleotide polymer, depending on context. From any specified polynucleotide sequence, either the given nucleic acid or the complementary polynucleotide sequence (e.g., the complementary nucleic acid) can be determined.

A “subsequence” is any portion of an entire sequence, up to and including the complete sequence. Typically a subsequence comprises less than the full-length sequence.

A “repeated sequence element” is a polynucleotide sequence that occurs in multiple copies in a particular organism's genome and/or in a sample of nucleic acid. Typically the repeated sequence element is present in multiple copies on a single chromosome or other single nucleic acid molecule. Repeated sequence elements can include imperfect or, more typically, perfect repeats. Repeated sequence elements of particular interest in the context of the present invention include those found as multiple tandem copies, e.g., with three or more copies of the repeated sequence element immediately adjacent to each other uninterrupted by any additional intervening polynucleotide sequence.

Two polynucleotides “hybridize” when they associate to form a stable duplex, e.g., under relevant assay conditions. Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology-Hybridization with Nucleic Acid Probes, part I chapter 2, “Overview of principles of hybridization and the strategy of nucleic acid probe assays” (Elsevier, New York), as well as in Ausubel, infra.

The “Tₘ” (melting temperature) of a nucleic acid duplex under specified conditions (e.g., relevant assay conditions) is the temperature at which half of the base pairs in a population of the duplex are disassociated and half are associated. The Tₘ for a particular duplex can be calculated and/or measured, e.g., by obtaining a thermal denaturation curve for the duplex (where the Tₘ is the temperature corresponding to the midpoint in the observed transition from double-stranded to single-stranded form).

The term “complementary” refers to a polynucleotide that forms a stable duplex with its “complement,” e.g., under relevant assay conditions. Typically, two polynucleotide sequences that are complementary to each other have mismatches at less than about 20% of the bases, at less than about 10% of the bases, preferably at less than about 5% of the bases, and more preferably have no mismatches.

A first polynucleotide that is “capable of hybridizing” (or, equivalently, “configured to hybridize”) to a second polynucleotide comprises a first polynucleotide sequence that is complementary to a second polynucleotide sequence in the second polynucleotide.

A “capture extender” or “CE” is a polynucleotide that is capable of hybridizing to a nucleic acid of interest, and that is preferably also capable of hybridizing to a capture probe. The capture extender typically has a first polynucleotide sequence C-1, which is complementary to the capture probe, and a second polynucleotide sequence C-3, which is complementary to a polynucleotide sequence of the nucleic acid of interest. Sequences C-1 and C-3 are typically not complementary to each other. The capture extender is preferably single-stranded.

A “capture probe” or “CP” is a polynucleotide that is capable of hybridizing to at least one capture extender and that is tightly bound (e.g., covalently or noncovalently, directly or through a linker, e.g., streptavidin-biotin or the like) to a solid support, a spatially addressable solid support, a slide, a particle, a microsphere, or the like. The capture probe typically comprises at least one polynucleotide sequence C-2 that is complementary to polynucleotide sequence C-1 of at least one capture extender. The capture probe is preferably single-stranded.

A “label extender” or “LE” is a polynucleotide that is capable of hybridizing to a nucleic acid of interest and to a label probe system. The label extender typically has a first polynucleotide sequence L-1, which is complementary to a polynucleotide sequence of the nucleic acid of interest, and a second polynucleotide sequence L-2, which is complementary to a polynucleotide sequence of the label probe system (e.g., L-2 can be complementary to a polynucleotide sequence of an amplification multimer, a preamplifier, a label probe, or the like). The label extender is preferably single-stranded.

A “label” is a moiety that facilitates detection of a molecule. Common labels in the context of the present invention include fluorescent, luminescent, light-scattering, and/or colorimetric labels. Suitable labels include enzymes and fluorescent moieties, as well as radionuclides, substrates, cofactors, inhibitors, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. Many labels are commercially available and can be used in the context of the invention.

A “label probe system” comprises one or more polynucleotides that collectively comprise one or more labels and one or more polynucleotide sequences M-1, each of which is capable of hybridizing to a label extender. The label provides a signal, directly or indirectly. Polynucleotide sequence M-1 is typically complementary to sequence L-2 in the label extenders. The one or more polynucleotide sequences M-1 are optionally identical sequences or different sequences. The label probe system can include a plurality of label probes (e.g., a plurality of identical label probes) and an amplification multimer; it optionally also includes a preamplifier or the like, or optionally includes only label probes, for example.

An “amplification multimer” is a polynucleotide comprising a plurality of polynucleotide sequences M-2, typically (but not necessarily) identical polynucleotide sequences M-2. Polynucleotide sequence M-2 is complementary to a polynucleotide sequence in the label probe. The amplification multimer also includes at least one polynucleotide sequence that is capable of hybridizing to a label extender or to a nucleic acid that hybridizes to the label extender, e.g., a preamplifier. For example, the amplification multimer optionally includes at least one polynucleotide sequence M-1; polynucleotide sequence M-1 is typically complementary to polynucleotide sequence L-2 of the label extenders. As another example, the amplification multimer optionally includes at least one polynucleotide sequence that is complementary to a polynucleotide sequence in a preamplifier (which in this example optionally includes at least one polynucleotide sequence M-1 that is complementary to polynucleotide sequence L-2 of the label extenders). The amplification multimer can be, e.g., a linear or a branched nucleic acid. As noted for all polynucleotides, the amplification multimer can include modified nucleotides and/or nonstandard internucleotide linkages as well as standard deoxyribonucleotides, ribonucleotides, and/or phosphodiester bonds. Suitable amplification multimers are described, for example, in U.S. Pat. No. 5,635,352, U.S. Pat. No. 5,124,246, U.S. Pat. No. 5,710,264, and U.S. Pat. No. 5,849,481.

A “label probe” or “LP” is a single-stranded polynucleotide that comprises a label (or optionally that is configured to bind to a label) that directly or indirectly provides a detectable signal. The label probe typically comprises a polynucleotide sequence that is complementary to the repeating polynucleotide sequence M-2 of the amplification multimer; however, if no amplification multimer is used in the bDNA assay, the label probe can, e.g., hybridize directly to a label extender.

A “preamplifier” is a nucleic acid that serves as an intermediate between one or more label extenders and amplification multimers. Typically, the preamplifier is capable of hybridizing simultaneously to at least one label extender and to a plurality of amplification multimers. The preamplifier can be, e.g., a linear or a branched nucleic acid.

As used herein, a “standard function” is a function, or expression of a function, that represents a relationship between two assay parameters, such as, e.g., a known assay input and the resulting output. The output can be a raw data output or a value (such as a number of molecules or concentration of an analyte) derived from the output. Standard functions and their expressions (e.g., standard curves) are well known in the art. The standard function can be in the form of an algebraic function (e.g., equation for a line) or can be provided in the form of a standard curve (e.g., resulting from regression analysis) on an X-Y chart. The standard function can also be expressed as a ratio, constant, or algorithm (e.g., in the form of computer software).

A “microsphere” is a small spherical, or roughly spherical, particle. A microsphere typically has a diameter less than about 1000 micrometers (e.g., less than about 100 micrometers, optionally less than about 10 micrometers).

A “microorganism” is an organism of microscopic or submicroscopic size. Examples include, but are not limited to, bacteria, fungi, yeast, protozoans, microscopic algae (e.g., unicellular algae), viruses (which are typically included in this category although they are incapable of growth and reproduction outside of host cells), subviral agents, viroids, and mycoplasma.

A variety of additional terms are defined or otherwise characterized herein.

### DETAILED DESCRIPTION

In one aspect, the present invention provides methods for analyzing repeated sequence elements, particularly tandem repeated sequence elements. The methods facilitate determination of the number of copies of the repeated sequence element present on a target nucleic acid molecule of interest and/or measurement of the length of the target nucleic acid occupied by copies of the repeated sequence element. The methods are useful for analyzing telomeric repeats, and thus for determining telomere length, either as an average for multiple chromosome arms (e.g., a genome average) or on a chromosome by chromosome or even arm by arm basis. The methods can also be applied to other repeated sequence elements, including but not limited to short tandem repeats (STRs, e.g., having 2-5 bp repeats), variable number of tandem repeats (VNTRs, e.g., having 9-80 bp core repeats), microsatellite repeats, minisatellite repeats, and trinucleotide repeats such as those found in Huntington's disease (CAG), fragile X syndrome (CGG), muscular atrophy (CAG), and myotonic dystrophy (CTG). Compositions, kits, and systems related to or useful in practicing the methods are also described.

In certain aspects, the methods and compositions for analyzing repeated sequence elements employ techniques and reagents similar to those employed in branched-chain DNA assays for nucleic acid detection. Accordingly, an overview of basic and multiplex branched-chain DNA assays is provided in the following section.

### Introduction to Branched-Chain DNA Assays

Branched-chain DNA (bDNA) signal amplification technology has been used, e.g., to detect and quantify mRNA transcripts in cell lines and to determine viral loads in blood. The bDNA assay is a sandwich nucleic acid hybridization procedure that enables direct measurement of mRNA expression, e.g., from crude cell lysate. It provides direct quantification of nucleic acid molecules at physiological levels. Several advantages of the technology distinguish it from other DNA/RNA amplification technologies, including linear amplification, good sensitivity and dynamic range, great precision and accuracy, simple sample preparation procedure, and reduced sample-to-sample variation.

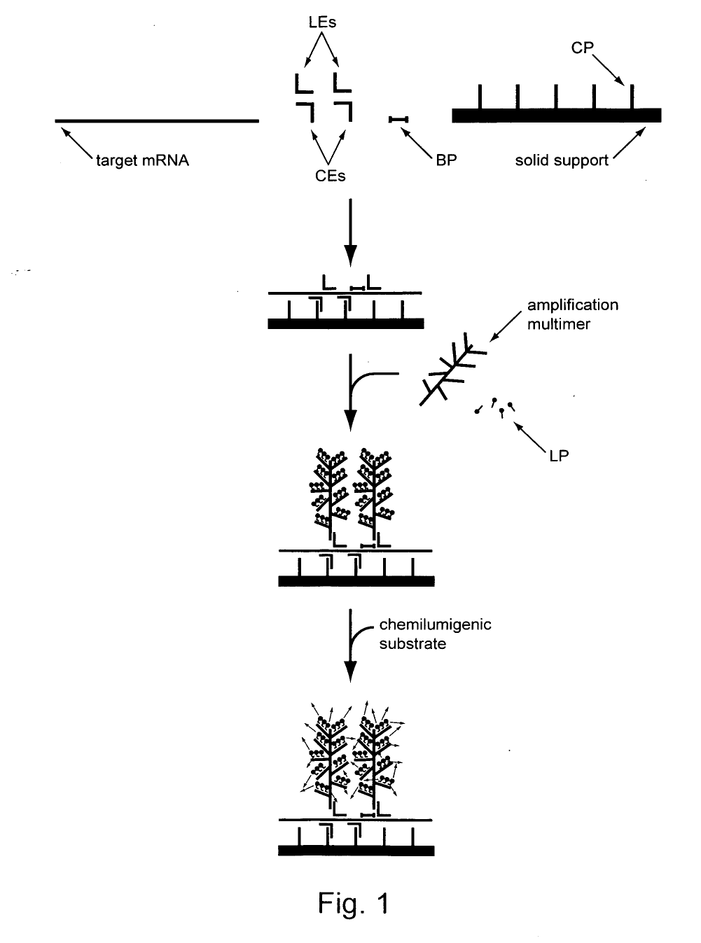

In brief, in a typical bDNA assay for gene expression analysis (schematically illustrated in FIG. 1), a target mRNA whose expression is to be detected is released from cells and captured by a Capture Probe (CP) on a solid surface (e.g., a well of a microtiter plate) through synthetic oligonucleotide probes called Capture Extenders (CEs). Each capture extender has a first polynucleotide sequence that can hybridize to the target mRNA and a second polynucleotide sequence that can hybridize to the capture probe. Typically, two or more capture extenders are used. Probes of another type, called Label Extenders (LEs), hybridize to different sequences on the target mRNA and to sequences on an amplification multimer. Additionally, Blocking Probes (BPs), which hybridize to regions of the target mRNA not occupied by CEs or LEs, are often used to reduce non-specific target probe binding. A probe set for a given mRNA thus consists of CEs, LEs, and optionally BPs for the target mRNA. The CEs, LEs, and BPs are complementary to nonoverlapping sequences in the target mRNA, and are typically, but not necessarily, contiguous. Probe set design confers specificity for the given mRNA.

Signal amplification begins with the binding of the LEs to the target mRNA. An amplification multimer is then typically hybridized to the LEs. The amplification multimer has multiple copies of a sequence that is complementary to a label probe (it is worth noting that the amplification multimer is frequently, but not necessarily, a branched-chain nucleic acid; for example, the amplification multimer can be a branched, forked, or comb-like nucleic acid or a linear nucleic acid). A label, for example, alkaline phosphatase, is covalently attached to each label probe. (Alternatively, the label can be noncovalently bound to the label probes.) In the final step, labeled complexes are detected, e.g., by the alkaline phosphatase-mediated degradation of a chemilumigenic substrate, e.g., dioxetane. Luminescence is reported as relative light unit (RLUs) on a microplate reader. The amount of chemiluminescence is proportional to the level of mRNA expressed from the target gene.

In the preceding example, the amplification multimer and the label probes comprise a label probe system. In another example, the label probe system also comprises a preamplifier, e.g., as described in U.S. Pat. No. 5,635,352 and U.S. Pat. No. 5,681,697, which further amplifies the signal from a single target mRNA. In this example, the LEs hybridize to sequences on the target mRNA and to the preamplifier, the preamplifier has multiple copies of a sequence that is complementary to the amplification multimer, and the amplification multimer has multiple copies of a sequence that is complementary to the label probe. Like the amplification multimer, the preamplifier can be, e.g., a branched, forked, comb-like, or linear nucleic acid. In yet another example, the label extenders hybridize directly to the label probes and no amplification multimer or preamplifier is used, so the signal from a single target mRNA molecule is only amplified by the number of distinct label extenders that hybridize to that mRNA.

Basic bDNA assays have been well described. See, e.g., U.S. Pat. No. 4,868,105 to Urdea et al. entitled “Solution phase nucleic acid sandwich assay”; U.S. Pat. No. 5,635,352 to Urdea et al. entitled “Solution phase nucleic acid sandwich assays having reduced background noise”; U.S. Pat. No. 5,681,697 to Urdea et al. entitled “Solution phase nucleic acid sandwich assays having reduced background noise and kits therefor”; U.S. Pat. No. 5,124,246 to Urdea et al. entitled “Nucleic acid multimers and amplified nucleic acid hybridization assays using same”; U.S. Pat. No. 5,624,802 to Urdea et al. entitled “Nucleic acid multimers and amplified nucleic acid hybridization assays using same”; U.S. Pat. No. 5,849,481 to Urdea et al. entitled “Nucleic acid hybridization assays employing large comb-type branched polynucleotides”; U.S. Pat. No. 5,710,264 to Urdea et al. entitled “Large comb type branched polynucleotides”; U.S. Pat. No. 5,594,118 to Urdea and Horn entitled “Modified N-4 nucleotides for use in amplified nucleic acid hybridization assays”; U.S. Pat. No. 5,093,232 to Urdea and Horn entitled “Nucleic acid probes”; U.S. Pat. No. 4,910,300 to Urdea and Horn entitled “Method for making nucleic acid probes”; U.S. Pat. No. 5,359,100; U.S. Pat. No. 5,571,670; U.S. Pat. No. 5,614,362; U.S. Pat. No. 6,235,465; U.S. Pat. No. 5,712,383; U.S. Pat. No. 5,747,244; U.S. Pat. No. 6,232,462; U.S. Pat. No. 5,681,702; U.S. Pat. No. 5,780,610; U.S. Pat. No. 5,780,227 to Sheridan et al. entitled “Oligonucleotide probe conjugated to a purified hydrophilic alkaline phosphatase and uses thereof”; U.S. patent application Publication No. US2002172950 by Kenny et al. entitled “Highly sensitive gene detection and localization using in situ branched-DNA hybridization”; Wang et al. (1997) “Regulation of insulin preRNA splicing by glucose” Proc Nat Acad Sci USA 94:4360-4365; Collins et al. (1998) “Branched DNA (bDNA) technology for direct quantification of nucleic acids: Design and performance” in Gene Quantification, F Ferre, ed.; Yao et al. (2004) “Multicenter Evaluation of the VERSANT Hepatitis B Virus DNA 3.0 Assay” J. Clin. Microbiol. 42:800-806; Elbeik et al. (2004) “Multicenter Evaluation of the Performance Characteristics of the Bayer VERSANT HCV RNA 3.0 Assay (bDNA)” J. Clin. Microbiol. 42:563-569; and Wilber and Urdea (1998) “Quantification of HCV RNA in clinical specimens by branched DNA (bDNA) technology” Methods in Molecular Medicine: Hepatitis C 19:71-78. In addition, kits for performing basic bDNA assays (QuantiGene° kits, comprising instructions and reagents such as amplification multimers, alkaline phosphatase labeled label probes, chemilumigenic substrate, capture probes immobilized on a solid support, and the like) are commercially available, e.g., from Affymetrix, Inc. (on the world wide web at www (dot) panomics (dot) com or www (dot) affymetrix (dot) com). Software for designing probe sets for a given mRNA target (i.e., for designing the regions of the CEs, LEs, and optionally BPs that are complementary to the target) is also available (e.g., ProbeDesigner™; see also Bushnell et al. (1999) “ProbeDesigner: for the design of probe sets for branched DNA (bDNA) signal amplification assays Bioinformatics 15:348-55).

The basic bDNA assay described above generally permits detection of a single target nucleic acid per assay. Multiplex bDNA assays for detection of two or more targets simultaneously have also been described. In brief, in an exemplary particle-based multiplex bDNA mRNA assay, different mRNAs are captured to different sets of microspheres. Each different mRNA is captured, through its own complementary set of CEs, to a distinguishable (e.g., fluorescently color-coded) set of microspheres bearing a CP complementary to that particular set of CEs. LEs and BPs are also hybridized to the mRNA targets, as for the singleplex assay described above. A label probe system (e.g., preamplifier, amplification multimer, and label probe) are then hybridized to the LEs as described above. Typically the label probe is fluorescently labeled (e.g., the LP can be biotinylated and detected with streptavidin conjugated phycoerythrin), and each set of microspheres is identified (e.g., by its unique fluorescence) and fluorescent emission by the label is measured for each set. The amount of label fluorescence for a given set of microspheres is proportional to the level of mRNA captured by that particular set of microspheres. A large number of mRNAs can be detected in a single reaction, e.g., 50 or more targets can be assayed using 50 or more different sets of microspheres.

For additional information relevant to multiplex assays, see commonly owned U.S. application publications 2006/0286583 entitled “Multiplex branched-chain DNA assays” by Luo et al., 2006/0263769 entitled “Multiplex capture of nucleic acids” by Luo et al., and 2007/0015188 entitled “Multiplex detection of nucleic acids” by Luo et al. See also Flagella et al. (2006) “A multiplex branched DNA assay for parallel quantitative gene expression profiling” Anal. Biochem. 352:50-60 and international application publication WO 2009/048530 by Martin, et al. entitled “Highly multiplexed particle-based assays.” QuantiGene® Plex kits for performing basic multiplex bDNA assays comprising instructions and reagents such as preamplifiers, amplification multimers, label probes, capture probes immobilized on microspheres, and the like are commercially available, e.g., from Affymetrix, Inc.

It will be evident that, in a bDNA assay, the degree of signal amplification depends on factors such as the composition of the label probe system and the number of label extenders that hybridize to a given target molecule. For example, in a system in which signal amplification involves sequential hybridization of a preamplifier having twenty repeats to which the amplification multimer can hybridize and an amplification multimer having twenty repeats (sequence M-2) to which the label probe can bind, signal amplification is 400-fold per label extender (i.e., 400 copies of the LP are captured per LE).

### Detection of Repeated Sequence Elements

In the bDNA assays for detection of mRNAs (or other nucleic acids) described above, for each given nucleic acid a set of several different LEs complementary to different regions of the nucleic acid is generally designed and hybridized to the nucleic acid to detect the presence and/or quantity of that nucleic acid in a sample. In contrast, in the methods described herein for detection of a repeated sequence element, only a single LE—complementary to the repeated sequence element or a subsequence thereof—is required. The number of copies of that single LE that hybridize to one copy of a nucleic acid of interest is proportional to the number of copies of the repeated sequence element present on that nucleic acid.

While the methods and compositions of the invention are generally applicable to any repeated sequence element, including, e.g., elements that are widely spaced on the nucleic acid target molecule and proximal elements (e.g., where the repeats are separated by 500 or fewer, 250 or fewer, 100 or fewer, 50 or fewer, 20 or fewer, or 10 or fewer intervening nucleotides), repeated sequence elements of greatest interest herein are tandemly repeated elements (e.g., where two or three or more repeats are immediately adjacent to each other).

Accordingly, a first general class of embodiments provides methods of detecting copy number of a repeated sequence element that is present in multiple tandem copies on a first nucleic acid target molecule. In the methods, the first nucleic acid target molecule (the nucleic acid of interest) is provided, e.g., by providing a test sample comprising the first nucleic acid target molecule. Multiple copies of a label extender are provided. Each copy of the label extender is capable of hybridizing to at least one copy of the repeated sequence element or to a subsequence thereof. A label probe system comprising a label, wherein a component of the label probe system is capable of hybridizing to the label extender, is also provided.

The label extender copies and the copies of the repeated sequence element or subsequence thereof on the first nucleic acid target molecule are hybridized, and the label probe system is hybridized to the label extender copies. The configuration of the label probe system can be varied, e.g., as described above. As one example, the label probe system can include a preamplifier, an amplification multimer, and a label probe, where the preamplifier hybridizes to a copy of the label extender and to a plurality of copies of the amplification multimer and each amplification multimer hybridizes to a plurality of copies of the label probe. A large number of copies of the label can thus be associated with each copy of the label extender, and thus with the nucleic acid target. As noted above, the degree of signal amplification is related to the number of label extenders that bind each target molecule and to the configuration of the label probe system (e.g., whether one or more preamplifier, an amplification multimer, and a label probe, or an amplification multimer and a label probe, or only a label probe is employed; the number of label probes bound by each amplification multimer; the number of amplification multimers bound by each preamplifier; the number of labels per label probe, and the like).

A signal from the label (e.g., a fluorescent, luminescent, or other optical signal) is detected, and its intensity is correlated with a number of copies of the repeated sequence element and/or with a length of the first nucleic acid target molecule occupied by the copies of the repeated sequence element. Since the number of label extenders that bind each target molecule is proportional to the number of copies of the repeated sequence element on the target molecule, the intensity of the signal is proportional to the element's copy number. It will be evident that copy number of the repeated sequence element and the length occupied by the element are different ways of expressing equivalent information, since the length of the target molecule occupied by the element equals the element's copy number times the length of the element (which is generally previously known). For example, for a 6 bp telomeric repeat, where 500 copies are detected the telomere length is 3 kb; similarly, where the telomere length is measured as 3 kb, 500 copies are present.

The methods are optionally performed with the nucleic acid target inside a cell or free in solution. Typically, however, the first nucleic acid target molecule is captured on a solid support, e.g., prior to detecting the signal from the label. For example, the target can be captured to the support by direct binding (covalent or noncovalent) to the support. More typically, however, the target is captured using oligonucleotides that are in turn bound, directly or indirectly, to the support.

Thus, in one class of embodiments, the first nucleic acid target molecule is captured on the solid support by providing a first set of one or more capture extenders, which first set of capture extenders is capable of hybridizing to the first nucleic acid target molecule, hybridizing the first set of capture extenders to the first nucleic acid target molecule, and associating the first set of capture extenders with the solid support, whereby hybridizing the first set of capture extenders to the first nucleic acid target molecule and associating the first set of capture extenders with the solid support captures the first nucleic acid target molecule on the solid support. The capture extenders are optionally bound to the solid support, e.g., covalently or noncovalently, directly or through a linker, e.g., streptavidin-biotin or the like. In a preferred aspect, the capture extenders are associated with the solid support by hybridization of the capture extenders to one or more capture probes. Thus, in one class of embodiments, a first capture probe is bound to the solid support, and the first set of capture extenders is associated with the solid support by hybridizing the capture extenders to the first capture probe.

As noted, the first set of capture extenders includes one or more capture extenders. In some embodiments, the set includes more than one CE, e.g., two, three, four, or five or more CEs. To facilitate capture of as much of the target nucleic acid as possible from the sample, more than 10 CEs per set can be employed, e.g., between 20 and 50 or even more. In embodiments in which the first set includes two or more capture extenders, the capture extenders in the first set preferably hybridize to nonoverlapping polynucleotide sequences in the first nucleic acid target molecule. The nonoverlapping polynucleotide sequences can, but need not be, consecutive within the first nucleic acid target. For specific capture of the first nucleic acid target, the capture extenders are preferably complementary to one or more sequences unique to the target rather than shared with other nucleic acids in the sample. In such embodiments, the capture extenders preferably hybridize to one or more polynucleotide sequences in the first nucleic acid target molecule other than the repeated sequence element or a subsequence thereof, and thus do not compete with the label extender for binding to the target nucleic acid.

In some embodiments, the first set of capture extenders includes a single capture extender. For example, in one class of embodiments, multiple copies of a single capture extender that hybridizes to at least one copy of the repeated sequence element or to a subsequence thereof are provided (e.g., sequences L-1 and C-3 can be identical). In such embodiments, the label extender is optionally provided in excess of the capture extender. For example, the label extender:capture extender ratio can be between 1:10 and 10:1, e.g., between 2:10 and 10:1, e.g., 4:1. In embodiments employing multiple copies of a single capture extender, the assay is typically configured such that hybridization of a single capture extender to the target nucleic acid and to the capture probe is not sufficient to stably capture the nucleic acid to the solid support. For example, hybridization of the capture extender to the capture probe and the target can be performed at a hybridization temperature that is greater than a melting temperature Tₘ of a complex between the individual capture extender and the capture probe. For additional details on assays requiring cooperative hybridization of two or more capture extenders to capture the target to the support, see, e.g., U.S. application publication 2006/0286583 entitled “Multiplex branched-chain DNA assays” by Luo et al. As another example, a single capture extender capable of hybridizing to a different repeated sequence element can be provided.

In embodiments in which a first capture probe is employed, each capture extender in the first set is capable of hybridizing to the first capture probe. The capture extender typically includes a polynucleotide sequence C-1 that is complementary to a polynucleotide sequence C-2 in the capture probe. The capture probe can include polynucleotide sequence in addition to C-2, or C-2 can comprise the entire polynucleotide sequence of the capture probe. For example, each capture probe optionally includes a linker sequence between the site of attachment of the capture probe to the solid support and sequence C-2 (e.g., a linker sequence containing 8 Ts, as just one possible example). Typically, each capture probe includes a single sequence C-2, and each capture extender in the first set includes the same nucleotide sequence as its sequence C-1. A number of other configurations are contemplated, however; for example, the capture probe can include two or more sequences C-2 (of the same or different nucleotide sequence), different capture extenders can include different nucleotide sequences as their sequence C-1, complementary to different sequences C-2 in a single or in different first capture probes, and the like.

The solid support can be essentially any suitable support, including any of a variety of materials, configurations, and the like. For example, in one class of embodiments, the solid support is a substantially planar solid support, typically rigid and optionally spatially addressable, e.g., an upper surface of the bottom of a well of a multiwell plate, a slide, or the like. Similarly, suitable solid supports include any surface of a well of a multiwell plate, whether planar or not. As another example, the solid support can comprise a plurality of particles, e.g., microspheres, beads, cylindrical particles, irregularly shaped particles, or the like. The particles are optionally identifiable, as will be described in greater detail below, and optionally have additional or other desirable characteristics. For example, the particles can be magnetic or paramagnetic, providing a convenient means for separating the particles from solution, e.g., to simplify separation of the particles from any materials not bound to the particles. Exemplary materials for the solid support include, but are not limited to, glass, silicon, silica, quartz, plastic, polystyrene, nylon, and nitrocellulose.

As noted above, the label probe system optionally includes an amplification multimer and a plurality of label probes, wherein the amplification multimer is capable of hybridizing to a label extender and to a plurality of label probes. In another aspect, the label probe system includes a preamplifier, a plurality of amplification multimers, and a plurality of label probes, wherein the preamplifier hybridizes to the label extenders, and the amplification multimers hybridize to the preamplifier and to the plurality of label probes. As another example, the label probe system can include only label probes, which hybridize directly to the label extenders. In one class of embodiments, the label probe comprises the label. In other embodiments, the label probe is configured to bind a label; for example, a biotinylated label probe can bind to a streptavidin-associated label.

The label can be essentially any convenient label that directly or indirectly provides a detectable signal. In one aspect, the label is a fluorescent label (e.g., a fluorophore or quantum dot). Detecting the signal from the label thus comprises detecting a fluorescent signal from the label. Fluorescent emission by the label is typically distinguishable from fluorescent emission by any particles employed as a solid support, e.g., microspheres, and many suitable fluorescent label-fluorescent microsphere combinations are possible. As other examples, the label can be a luminescent label, a light-scattering label (e.g., colloidal gold particles), or an enzyme (e.g., HRP or alkaline phosphatase).

The various hybridization and association steps in the methods can, e.g., be either simultaneous or sequential, in essentially any convenient order. At any of various steps, materials not associated with the nucleic acid target are optionally separated from the target. For example, in one exemplary embodiment in which the nucleic acid target is bound to a solid support, after the nucleic acid target, label extender copies, and optional capture extenders, blocking probes, and support-bound capture probes are hybridized, the support is optionally washed to remove unbound nucleic acids and probes; after the label extender copies and preamplifier or amplification multimer are hybridized, the support is optionally washed to remove unbound preamplifier or amplification multimer; and/or after the label probes are hybridized to the amplification multimer, the support is optionally washed to remove unbound label probe prior to detection of the label.

The first nucleic acid target molecule can be essentially any desired nucleic acid, including but not limited to, DNA, RNA, eukaryotic, bacterial and/or viral genomic RNA and/or DNA (double-stranded or single-stranded), and extra-genomic DNA. In one class of embodiments, the first nucleic acid target molecule comprises a chromosome or portion thereof, for example, a chromosome or portion thereof from a eukaryote (e.g., a plant, animal, vertebrate, human, insect, protist, fungus, yeast, or cultured cell). For example, in embodiments in which telomere length is to be analyzed, the first nucleic acid target molecule can comprise a distal portion of a chromosome arm (e.g., the portion of the chromosome arm, e.g., of the left or right arm, that is furthest from the centromere and that includes the telomere) and the repeated sequence element can be a telomeric repeat.

It will be understood that if the first nucleic acid target is initially present in the sample in a double-stranded form, e.g., hybridized to a complementary nucleic acid, the double-stranded form is typically denatured prior to hybridizing the first target nucleic acid to the label extender and optional first set of capture extenders. Denaturation can be accomplished, for example, by thermal denaturation, exposure to alkaline conditions (which can have the added advantage of digesting extraneous RNA if the nucleic acid target is a DNA), or similar techniques. The methods can thus be used for detecting repeated sequence elements on, e.g., double-stranded genomic DNA, double-stranded viral nucleic acids, and the like, as well as on single-stranded nucleic acids. Very long nucleic acids, such as chromosomes, are optionally fragmented to ease handling, e.g., by shearing, restriction enzyme digestion, etc. prior to the assay. Salt concentration can be adjusted to increase stability and prevent undesired degradation of long nucleic acids, e.g., chromosome arms, prior to or during the assay.

As noted above, exemplary repeated sequence elements of particular interest include telomeric repeats. In one class of embodiments, the telomeric repeat is TTAGGG, or its complement CCCTAA, depending on which strand of the chromosome is desirably analyzed. Other exemplary telomeric repeats are noted above, and a large number of additional telomeric repeat sequences have been determined and are available in the literature or can be determined using known techniques.

Other repeated sequence elements of particular interest include those whose copy numbers are altered in disease states. For example, Huntington's disease is associated with expansion of a CAG repeat in the coding region of the human IT15 gene, typically from less than 20 copies in unaffected individuals to more than 30, e.g., more than 35, in affected individuals (Squitieri et al. (2003) “Homozygosity for CAG mutation in Huntington disease is associated with a more severe clinical course” Brain 126:946-955). Similar expansions of trinucleotide repeats occur in other conditions, such as fragile X syndrome (CGG), muscular atrophy (CAG), and myotonic dystrophy (CTG) (see, e.g., U.S. Pat. No. 5,962,332). As another example, different numbers of a VNTR at the 5′ flanking end of the insulin gene has been associated with diabetes; e.g., alleles with about 40 VNTR elements related to consensus sequence ACAGGGGTGTGGGG (SEQ ID NO:1) are associated with type I diabetes susceptibility while alleles with more than 100 repeat elements are not (Owerbach and Gabbay (1994) “Linkage of the VNTR/insulin-gene and type I diabetes mellitus: increased gene sharing in affected sibling pairs” Am J Hum Genet 54:909-912; Lew et al. (2000) “Unusual DNA structure of the diabetes susceptibility locus IDDM2 and its effect on transcription by the insulin promoter factor Pur-1/MAZ” Proc Natl Acad Sci 97:12508-12512). Thus, as just two examples, for detection of Huntington's disease alleles exemplary label extenders include 5-8 or more copies of CAG or its complement CTG and exemplary capture extenders include other IT15 gene sequences, while for detection of diabetes-associated alleles exemplary label extenders include two copies of the relevant VNTR or its complement and exemplary capture extenders include other insulin gene sequences.

The methods of the invention can also be applied to tetranucleotide repeats and other STRs used in forensics, such as those included in the U.S. national CODIS database. Additional repeated sequence elements of interest in the context of the present invention include, but are not limited to, other short tandem repeats (STRs, e.g., having 2-5 bp repeats), variable number of tandem repeats (VNTRs, e.g., having 9-80 bp core repeats), microsatellite repeats, and minisatellite repeats. Note that where a polynucleotide sequence for a repeated sequence element is indicated, either the noted sequence or equivalently its complement on the opposite strand can optionally be detected, as convenient and desirable for the particular application of interest.

As indicated above, repeated sequence elements of greatest interest herein are tandemly repeated elements, where multiple (at least two, e.g., 3, 4, or 5 or more) repeats are immediately adjacent to each other. Typically, the repeated sequence element is present in at least 10 tandem copies on the first nucleic acid target molecule, and more typically in at least 20 tandem copies, at least 30 tandem copies, at least 40 tandem copies, at least 50 tandem copies, or at least 100 tandem copies. Optionally, the repeated sequence element is present in at least 250, at least 500, at least 1000, at least 2000, or even at least 3000 tandem copies on the first nucleic acid target molecule.

The repeated sequence element to be analyzed can be essentially any desired repeated element of any length (e.g., 500 nucleotides or less, 250 nucleotides or less, 200 nucleotides or less, 150 nucleotides or less, or 100 nucleotides or less in length). More typically, however, each copy of the repeated sequence element is 50 nucleotides or less in length, for example, 25 nucleotides or less, 24 nucleotides or less, 22 nucleotides or less, 20 nucleotides or less, 15 nucleotides or less, or even 10 nucleotides or less in length. The methods are applicable even to short tandem repeats difficult to assay by other techniques, including repeats having 7, 6, 5, 4, 3, and 2 nucleotides.

Depending, e.g., on the length of the repeated sequence element, the label extender can hybridize to a subsequence of the element (e.g., for longer elements), to the entirety of a single copy of the element, or to at least two tandem copies of the repeated sequence element (e.g., for shorter elements). Optionally, the label extender is capable of hybridizing to at least three, four, five, or more tandem copies of the repeated sequence element. For example, for detection of the 6 bp vertebrate telomeric repeat TTAGGG, the label extender optionally hybridizes to three or four tandem TTAGGG repeats (that is, the label extender includes CCCTAACCCTAACCCTAA (SEQ ID NO:2) or CCCTAACCCTAACCCTAACCCTAA (SEQ ID NO:3) as polynucleotide sequence L-1). As other examples, for detection of a tetranucleotide repeat the LE optionally hybridizes to six tandem repeats, and for detection of a trinucleotide repeat the LE optionally hybridizes to eight tandem repeats.

Typically, at least two identical copies of the label extender hybridize to a single copy of the target molecule. It is worth noting that the number of copies of the label extender that hybridize to the target molecule is not necessarily equal to the number of copies of the repeated sequence element on the target. In embodiments in which the label extender is complementary to a subsequence of the repeated sequence element, for example, a number of copies of the label extender up to the number of copies of the repeated sequence element can hybridize to the target. Similarly, in embodiments in which the label extender is complementary to the entirety of a single copy of the repeated sequence element, a number of copies of the label extender up to the number of copies of the repeated sequence element on the target can hybridize to the target. However, it will be evident that, in embodiments in which the label extender is capable of hybridizing to two or more tandem copies of the repeated sequence element, the number of label extender copies will be less than the number of copies of the repeated sequence element. (As just one example, where the label extender is complementary to four tandem copies of the repeated sequence element and the nucleic acid target includes 100 copies of the element, up to 25 copies of the label extender can hybridize to the target molecule.) It is worth noting that the maximum possible number of label extender copies may not always hybridize to the target, e.g., if the label extender copies do not align precisely in register (e.g., in the preceding example, 1-3 copy gaps may be left between adjacent hybridized label extender copies), but this is not expected to significantly influence the results of the assay.

The methods can be conveniently multiplexed to analyze the repeated sequence element on two or more nucleic acid molecules simultaneously. Thus, in one class of embodiments, the test sample also comprises a second nucleic acid target molecule that is distinct from the first nucleic acid target molecule and that comprises multiple, typically tandem, copies of the repeated sequence element. The methods include hybridizing the label extender copies to the copies of the repeated sequence element or subsequence thereof on the second nucleic acid target molecule, e.g., in the same reaction mixture and at the same time that other label extender copies are hybridized to the repeated sequence element copies on the first nucleic acid target. The label probe system is hybridized to the label extenders and signal is detected as described above. Third, fourth, fifth, etc. (or even tenth, twentieth, fiftieth, hundredth, etc.) nucleic acid target molecules comprising the repeated sequence element are optionally included in the test sample and detected with the label extender as noted for the second target.

The first and second (and optional third, fourth, etc.) nucleic acid target molecules are optionally captured on a solid support, e.g., prior to or simultaneous with hybridization of the label extender copies to the repeated sequence element copies and prior to detection of the signal. If an average repeated sequence element copy number or length occupied by the element is desired for the two (three, four, etc.) target molecules, then the molecules can be captured in a single well of a multiwell plate, on a single spot on an array, on a single set of particles, or the like. If the copy number or length occupied by the repeated sequence element on each separate molecule is desired, however, then different target molecules are conveniently captured at different positions in an array, on different distinguishable sets of particles, or the like.

Thus, in one class of embodiments, the solid support is a substantially planar solid support, and the first nucleic acid target molecule is captured at a first selected position on the solid support and the second nucleic acid target molecule is captured at a second selected position (different from the first) on the solid support. The signal from the label is then detected at each different selected position on the solid support. The intensity of the signal for a given position is correlated with the number of copies of the repeated sequence element on the corresponding nucleic acid target molecule and/or with the length of the corresponding nucleic acid target molecule occupied by the copies of the repeated sequence element (e.g., the intensity of the signal measured for the first position is correlated with the copy number or length for the first target molecule, that for the second with the second, etc.). Spatially addressable non-planar solid supports can optionally also be employed in the methods. The solid support can be essentially any suitable spatially addressable support, including any of a variety of materials, configurations, and the like, e.g., an upper surface of the bottom of a well of a multiwell plate, a slide, or the like.

In a similar class of embodiments, the solid support comprises a population of particles that includes at least two sets of particles, the particles in each set being distinguishable from the particles in every other set. The first nucleic acid target molecule is captured on a first set of the particles, and the second nucleic acid target molecule is captured on a second set of the particles. At least a portion of the particles from each set is identified, and the signal from the label on those particles is detected. The intensity of the signal for a given set of particles is correlated with the number of copies of the repeated sequence element on the corresponding nucleic acid target molecule and/or with the length of the corresponding nucleic acid target molecule occupied by the copies of the repeated sequence element (e.g., the intensity of the signal measured for the first set of particles is correlated with the copy number or length for the first target molecule, that for the second with the second, etc.).

Essentially any suitable particles, e.g., particles having distinguishable characteristics and to which capture probes can be attached, can be used. For example, in one preferred class of embodiments, the particles are microspheres. The microspheres of each set can be distinguishable from those of the other sets, e.g., on the basis of their fluorescent emission spectrum, their diameter, or a combination thereof. For example, the microspheres of each set can be labeled with a unique fluorescent dye or mixture of such dyes, quantum dots with distinguishable emission spectra, and/or the like. As another example, the particles of each set can be identified by an optical barcode, unique to that set, present on the particles. The particles optionally have additional or other desirable characteristics. For example, the particles can be magnetic or paramagnetic, providing a convenient means for separating the particles from solution, e.g., to simplify separation of the particles from any materials not bound to the particles.

The first, second, third, etc. nucleic acid targets are optionally captured as described for single targets above, e.g., using CEs and CPs, direct binding, or the like. Accordingly, in one exemplary class of embodiments, capturing the first nucleic acid target molecule on a solid support comprises providing a first set of one or more capture extenders, which first set of capture extenders is capable of hybridizing to the first nucleic acid target molecule, hybridizing the first set of capture extenders to the first nucleic acid target molecule, and associating the first set of capture extenders with the solid support, whereby hybridizing the first set of capture extenders to the first nucleic acid target molecule and associating the first set of capture extenders with the solid support captures the first nucleic acid target molecule on the solid support, and capturing the second nucleic acid target molecule on a solid support comprises providing a second set of one or more capture extenders, which second set of capture extenders is capable of hybridizing to the second nucleic acid target molecule, hybridizing the second set of capture extenders to the second nucleic acid target molecule, and associating the second set of capture extenders with the solid support, whereby hybridizing the second set of capture extenders to the second nucleic acid target molecule and associating the second set of capture extenders with the solid support captures the second nucleic acid target molecule on the solid support. It will be evident that the number of capture extenders in the first set and second sets can, but need not, be the same. It will also be evident that third, fourth, fifth, hundredth, etc. sets of capture extenders are optionally provided (optionally along with third, fourth, fifth, hundredth, etc. sets of particles or positions), depending on the number of nucleic acid targets of interest in the assay.

In embodiments in which an average copy number or length occupied by the repeated sequence element is desired and the first and second nucleic acid targets are thus captured together in a single location, the second set of capture extenders can be identical to the first set of capture extenders. For example, capture extenders can be designed to hybridize to Alu or alpha satellite or other repetitive sequences to capture essentially an entire vertebrate genome, e.g., such that an average telomere length can be measured for all the chromosomes. As another example, a single capture extender that, like the label extender, hybridizes to at least one copy of the repeated sequence element or a subsequence thereof can be used to capture all of the target nucleic acids.

In embodiments in which different target molecules are to be captured to different positions in an array or to different sets of particles for separate measurement of copy number or length, however, the first and second sets of capture extenders are different, specific for their respective targets. Optionally, in such embodiments, the first position on the solid support or first set of particles comprises a first capture probe, which first capture probe is capable of hybridizing to the capture extenders comprising the first set of capture extenders, and the second position on the support or set of particles comprises a distinct, second capture probe, which second capture probe is capable of hybridizing to the capture extenders comprising the second set of capture extenders. Each nucleic acid target can thus; by hybridizing to its corresponding set of capture extenders which are in turn hybridized to a corresponding capture probe, be associated with a selected position on a solid support or with an identifiable set of particles. Techniques for forming such arrays of capture probes are well known and are, e.g., referenced below in the sections entitled “Arrays” and “Microspheres.”

Blocking probes are optionally also hybridized to the nucleic acid target(s), which can reduce background in the assay. For a given nucleic acid target, the corresponding capture extenders, label extenders, and optional blocking probes are optionally complementary to physically distinct, nonoverlapping sequences in the nucleic acid of interest, which can but need not be contiguous. In other embodiments, as noted above, the label extender and capture extender can both bind to the repeated sequence element. The Tₘs of the capture extender-nucleic acid, label extender-nucleic acid, and blocking probe-nucleic acid complexes are preferably greater than the hybridization temperature, e.g., by 5° C. or 10° C. or preferably by 15° C. or more, such that these complexes are stable at the hybridization temperature. Potential CE and LE sequences (e.g., potential sequences C-3 and L-1) are optionally examined for possible interactions with non-corresponding nucleic acids, LEs or CEs, the amplification multimer, the preamplifier, the label probe, and/or any relevant genomic sequences, for example; sequences expected to cross-hybridize with undesired nucleic acids are typically not selected for use in the CEs or LEs. See, e.g., Player et al. (2001) “Single-copy gene detection using branched DNA (bDNA) in situ hybridization” J Histochem Cytochem 49:603-611. Examination can be, e.g., visual (e.g., visual examination for complementarity), computational (e.g., computation and comparison of binding free energies), and/or experimental (e.g., cross-hybridization experiments). Capture probe sequences are preferably similarly examined, to ensure that the polynucleotide sequence C-1 complementary to a particular capture probe's sequence C-2 is not expected to cross-hybridize with any of the other capture probes that are to be associated with other subsets of particles or positions on the support.

A capture probe and/or capture extender optionally comprises at least one non-natural nucleotide. For example, a capture probe and the corresponding capture extender optionally comprise, at complementary positions, at least one pair of non-natural nucleotides that base pair with each other but that do not Watson-Crick base pair with the bases typical to biological DNA or RNA (i.e., A, C, G, T, or U). Examples of nonnatural nucleotides include, but are not limited to, Locked NucleicAcid™ nucleotides (available from Exiqon A/S, (www(dot) exiqon (dot) com; see, e.g., SantaLucia Jr. (1998) Proc Natl Acad Sci 95:1460-1465) and isoG, isoC, and other nucleotides used in the AEGIS system (Artificially Expanded Genetic Information System, available from EraGen Biosciences, (www (dot) eragen (dot) com; see, e.g., U.S. Pat. No. 6,001,983, U.S. Pat. No. 6,037,120, and U.S. Pat. No. 6,140,496). Use of such non-natural base pairs (e.g., isoG-isoC base pairs) in the capture probes and capture extenders can, for example, reduce background and/or simplify probe design in multiplex assays by decreasing cross hybridization, or it can permit use of shorter CPs and CEs when the non-natural base pairs have higher binding affinities than do natural base pairs. Non-natural nucleotides can similarly be included in the label extenders, preamplifiers, amplification multimers, and/or label probes, if desired.

The methods can optionally be multiplexed for detection of different repeated sequence elements, e.g., on the same and/or different target molecules. For example, a first repeated sequence element can be detected on a first nucleic acid target molecule using copies of a first label extender as described above, while a second repeated sequence element of different sequence can be detected on a second nucleic acid target molecule using copies of a second label extender complementary to the second element, where the different targets are captured to different positions on a solid support or different sets of particles. As another example, first and second repeated sequence elements can be detected on one or more nucleic acid targets by employing two differently labeled label probe systems, one of which hybridizes to a label extender that recognizes the first repeated sequence element and the other of which hybridizes to a label extender that recognizes the second repeated sequence element.

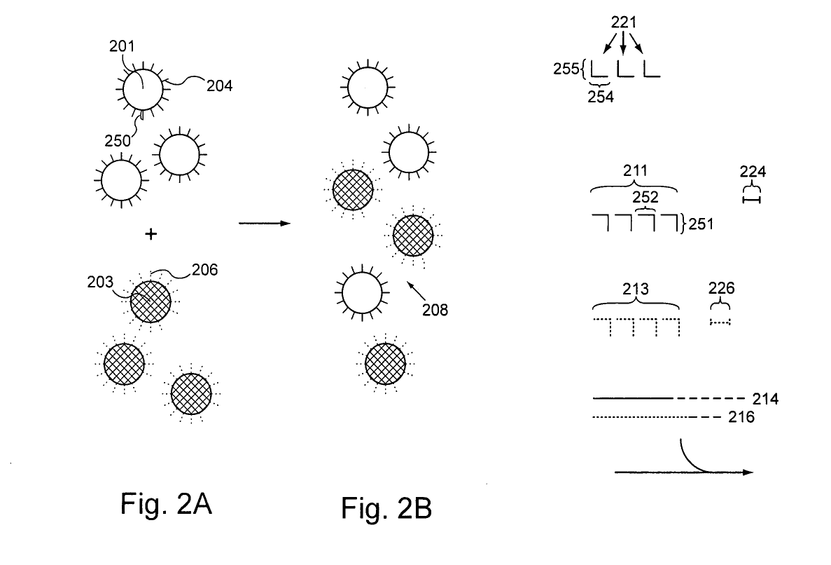

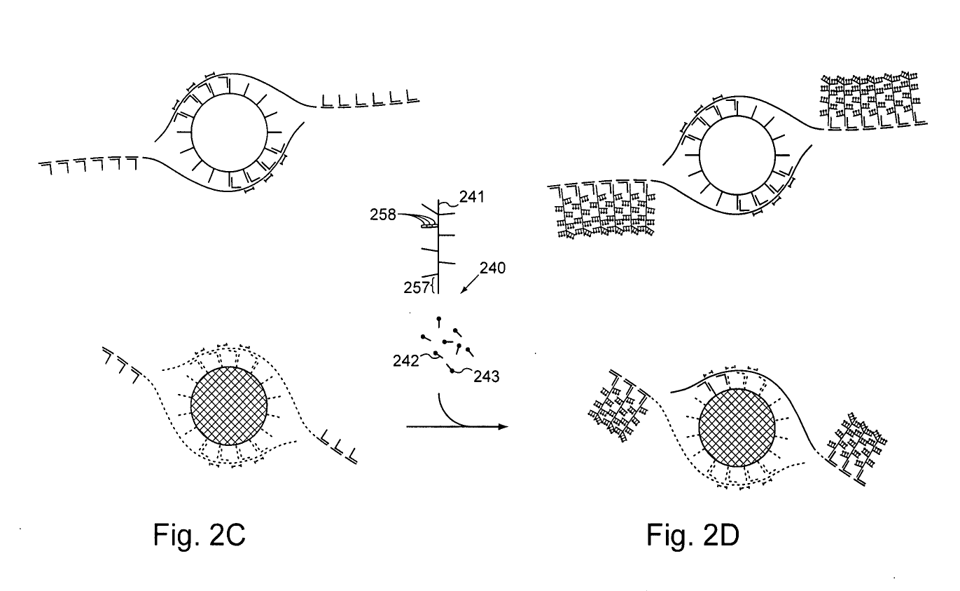

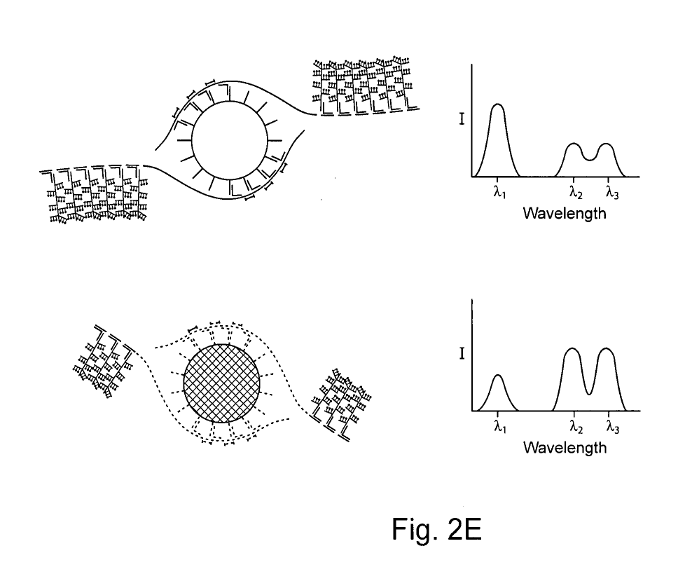

An exemplary embodiment illustrating multiplex detection of a repeated sequence element on multiple nucleic acid targets simultaneously is schematically illustrated in FIG. 2. In this example, the repeated sequence element is a telomeric repeat, and the telomere length of two different nucleic acid target molecules (two different chromosomes or even two different distal arm portions) is analyzed. Panel A illustrates two distinguishable subsets of microspheres 201 and 203, which have associated therewith capture probes 204 and 206, respectively. Each capture probe includes a sequence C-2 (250), which is different from subset to subset of microspheres. The two subsets of microspheres are combined to form pooled population 208 (Panel B). A subset of capture extenders is provided for each nucleic acid target molecule: subset 211 for chromosome arm 214 and subset 213 for chromosome arm 216. Each capture extender includes sequences C-1 (251, complementary to the respective capture probe's sequence C-2) and C-3 (252, complementary to a sequence in the corresponding nucleic acid target). Multiple copies of label extender 221 are provided. Label extender 221 includes sequences L-1 (254, complementary to the telomeric repeat or a portion thereof, e.g., complementary to four tandem copies of the telomeric repeat) and L-2 (255, complementary to M-1). Two subsets of blocking probes (224 and 226 for nucleic acids 214 and 216, respectively) are also provided.

Nucleic acids 214 and 216 are hybridized to their corresponding subset of capture extenders (211 and 213, respectively), and the capture extenders are hybridized to the corresponding capture probes (204 and 206, respectively), capturing nucleic acids 214 and 216 on microspheres 201 and 203, respectively (Panel C). Label extender 221 is hybridized to the telomeric repeat (e.g., one copy of the label extender to four tandem copies of the telomeric repeat). Materials not bound to the microspheres (e.g., extraneous nucleic acids, other chromosome arms, unbound CEs or LEs, etc.) are optionally separated from the microspheres by washing. Label probe system 240 including amplification multimer 241 (which includes sequences M-1 257 and M-2 258) and label probe 242 (which contains label 243) is hybridized to label extender 221 (Panel D). Materials not captured on the microspheres are optionally removed by washing the microspheres. Microspheres from each subset are identified, e.g., by their fluorescent emission spectrum (λ₂ and λ₃, Panel E), and signal from the label on each subset of microspheres is detected (λ₁, Panel E). Since each nucleic acid target is associated with a distinct subset of microspheres, the intensity of the signal from the label on a given subset of microspheres correlates with the telomere length of the corresponding nucleic acid target molecule. Microspheres from subset 201 thus display a more intense label signal than do those from subset 203, since the telomere length/telomeric repeat copy number is greater for chromosome arm 214 than for 216.

As depicted in FIG. 2, all of the label extenders typically include an identical sequence L-2. Optionally, however, different label extenders (e.g., label extenders in different subsets where different repeated sequence elements are to be detected) can include different sequences L-2. Also as depicted in FIG. 2, each capture probe typically includes a single sequence C-2 and thus hybridizes to a single capture extender. Optionally, however, a capture probe can include two or more sequences C-2 and hybridize to two or more capture extenders. Similarly, as depicted, each of the capture extenders in a particular subset typically includes an identical sequence C-1, and thus only a single capture probe is needed for each subset of particles; however, different capture extenders within a subset optionally include different sequences C-1 (and thus hybridize to different sequences C-2, within a single capture probe or different capture probes on the surface of the corresponding subset of particles). As noted, the label probe can include the label (e.g., a fluorescent label as in this example), or it can be configured to bind the label (e.g., the label probe can be biotinylated and bound by streptavidin conjugated phycoerythrin or other fluorophore).

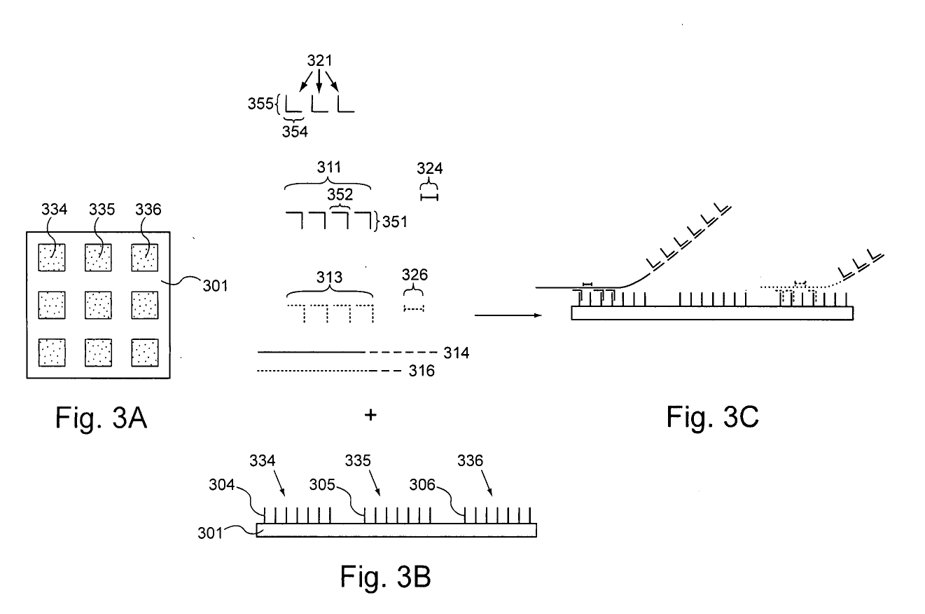

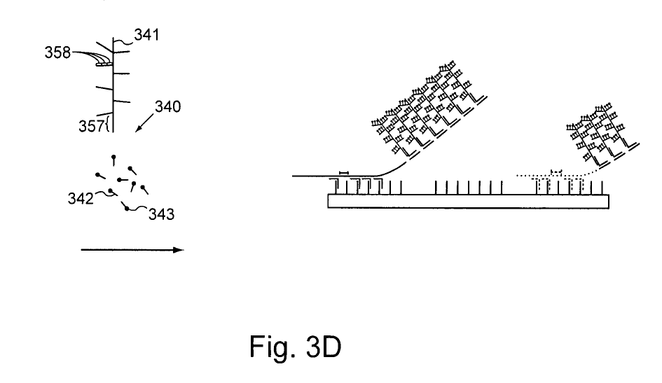

The preceding embodiment includes capture of the nucleic acid targets on particles. As an alternative, the nucleic acids can be captured at different positions on a non-particulate, spatially addressable solid support. An exemplary embodiment in which the repeated sequence element is a telomeric repeat and the telomere length of different chromosome arms is analyzed is schematically illustrated in FIG. 3. Panel A depicts solid support 301 having nine capture probes provided on it at nine selected positions (e.g., 334-336). Panel B depicts a cross section of solid support 301, with distinct capture probes 304, 305, and 306 at different selected positions on the support (334, 335, and 336, respectively). A subset of capture extenders is provided for each nucleic acid target molecule. Only two subsets are depicted; subset 311 for chromosome arm 314 and subset 313 for chromosome arm 316. Each capture extender includes sequences C-1 (351, complementary to the respective capture probe's sequence C-2) and C-3 (352, complementary to a sequence in the corresponding nucleic acid target molecule). Multiple copies of label extender 321 are provided. Label extender 321 includes sequences L-1 (354, complementary to the telomeric repeat or a subsequence thereof, e.g., complementary to four tandem copies of the repeat) and L-2 (355, complementary to M-1). Two subsets of blocking probes (324 and 326 for nucleic acids 314 and 316, respectively) are also depicted (although in an assay for telomere length of nine different arms, nine would typically be provided, one for each of the different chromosome arms).

Nucleic acids 314 and 316 are hybridized to their corresponding subset of capture extenders (311 and 313, respectively), and the capture extenders are hybridized to the corresponding capture probes (304 and 306, respectively), capturing nucleic acids 314 and 316 at selected positions 334 and 336, respectively (Panel C). Label extender 321 is hybridized to the telomeric repeat (e.g., one copy of the label extender to four tandem copies of the telomeric repeat). Materials not bound to the solid support (e.g., other chromosome arms, extraneous nucleic acids, unbound CEs or LE copies, etc.) are optionally separated from the support by washing. Label probe system 340 including amplification multimer 341 (which includes sequences M-1 357 and M-2 358) and label probe 342 (which contains label 343) is hybridized to label extender 321 (Panel D). Materials not captured on the solid support are optionally removed by washing the support, and signal from the label at each position on the solid support is detected. Since each nucleic acid target (chromosome arm) is associated with a distinct position on the support, the intensity of the signal from the label at a given position on the support correlates with the telomere length/telomeric repeat copy number of the corresponding chromosome arm.

It will be evident that, while the preceding two examples illustrate detection of telomere length on two chromosome arms, the methods can readily be extended to essentially any desired number of chromosomes and/or arms. For example, for telomere length measurement of each individual chromosome arm for an organism having n chromosomes in its haploid genome and therefore (at least) 2n different chromosome arms, the 2n different arms can be captured to 2n different positions on a solid support or to 2n different sets of particles, e.g., using a set of capture extenders designed specifically for each strand of each chromosome at the closest convenient region to the telomere. For humans, for example, the telomere length of each arm of all 23 chromosomes can be measured in parallel in a single assay with 46 microsphere sets or positions (48 if the Y chromosome is considered).

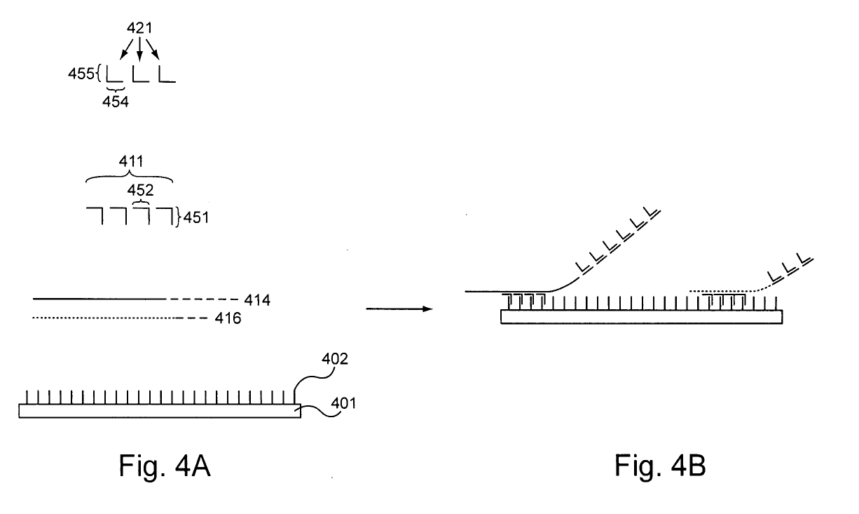

The preceding two examples illustrate multiplex detection of telomere length (and therefore multiplex detection of a repeated sequence element) on individual different nucleic acid targets. As indicated above, the methods can also be employed for detection of average telomere length (average copy number of or length occupied by a repeated sequence element) over different nucleic acid targets. An exemplary embodiment in which average telomere length is to be determined is schematically illustrated in FIG. 4. Panel A depicts solid support 401 having capture probe 402 provided on it. Subset 411 of capture extenders is provided. Each capture extender includes sequences C-1 (451, complementary to capture probe 402's sequence C-2) and C-3 (452, complementary to a sequence present in both chromosome arm 414 and chromosome arm 416). While in this example one set of capture extenders complementary to sequences found on both arms (e.g., Alu, alpha satellite, etc.) is employed, it will be evident that a mixture of different sets of capture extenders for the different chromosome arms can instead be employed if desired, as can a single capture extender complementary to a sequence of the telomeric repeat (e.g., to four tandem copies of the repeat). Multiple copies of label extender 421, including sequences L-1 (454, complementary to a sequence in the telomeric repeat, e.g., to four tandem copies of the repeat) and L-2 (455, complementary to M-1) are provided.

Chromosome arms 414 and 416 are hybridized to set 411 of capture extenders and the capture extenders are hybridized to capture probe 402. A mixture of chromosome arms 414 and 416 is thus captured to support 401. Unbound materials are optionally separated by washing, a label probe system is hybridized to the label extender, unbound materials are optionally separated by washing, and signal from the label on the solid support is detected. Since a mixture of the two chromosome arms is captured on the support, the intensity of the signal from the label is correlated with the average of the telomere length on the two arms. As for the examples above, the methods are readily extended to more than two arms (e.g., 46 or 48 to determine average telomere length for all human chromosomes).

The copy number of and/or length occupied by the repeated sequence element obtained by the methods is optionally expressed in relative or absolute terms. The initial output value of an assay is typically in some unit of magnitude, such as, e.g., absorbance units, fluorescence units, relative light units (RLU), a voltage, a light intensity, a radioactive particle count, or the like. In one aspect, the intensity of the signal for a given target (or for a given sample, in embodiments in which an average value for multiple targets is assayed instead of a value for each individual target) is compared to that for a reference or control. As one example, in an embodiment in which telomere length of human chromosomes is being assayed, the intensity of the signal from a biopsy sample can be compared to that from an equivalent amount of normal healthy tissue (whether for a single arm or multiple arms). As another example, in an embodiment in which trinucleotide repeat copy number is being assayed in an individual suspected of carrying Huntington's disease, intensity can be compared with that from an equivalent number of cells from an unaffected individual. Copy number of a repeated sequence element or length occupied by the element can thus be expressed in relative terms, e.g., more or fewer, or longer or shorter, than a control.

In another aspect, the intensity value can be input to a standard function to output a defined quantity, e.g., a number of copies, number of nucleotides (length), mass, etc. For example, a standard function can be established to represent the relationship between the input of a known number of copies of the repeated sequence element or a known length occupied by the element and the output intensity of an assay according to the methods. The intensity measured for a given target (or targets) in a test sample can thus be converted to a number of copies or length occupied, using the standard function.

If desired, the copy number of or length occupied by the repeated sequence element can be expressed as a number of copies or length per target molecule, per chromosome, per cell, per μg of nucleic acid (e.g., target nucleic acid or total cellular nucleic acid) in the test sample, or the like. One approach to doing so involves normalizing the intensity of the signal, or the copy number or length computed from the intensity, by comparison with a reference nucleic acid. Accordingly, in one class of embodiments a standard function for cell number or amount of cellular nucleic acid input versus quantity of a reference nucleic acid is provided. The reference nucleic acid is quantitated from the test sample. A cell number or amount of cellular nucleic acid is determined for the test sample based on the standard function and the quantity of reference nucleic acid in the test sample, and the intensity of the signal, the number of copies, and/or the length is normalized to the cell number or amount of cellular nucleic acid. In another class of embodiments, the intensity of the signal is simply normalized to an intensity measured for a reference nucleic acid (and optionally compared to normalized signal from a reference or control sample).

Suitable reference nucleic acids generally include those present in multiple copies and typically at fairly high and stable copy numbers. Exemplary reference nucleic acids include, but are not limited to, a ribosomal DNA (e.g., an 18S rDNA, 5.8S rDNA, or 28S rDNA), an Alu sequence, and a β-globin gene.

Use of standard functions is briefly summarized using cell number and an rDNA reference nucleic acid by way of example; it will be evident similar standard functions are readily derived for chromosome number, amount (e.g., mass or concentration) of nucleic acid, other reference nucleic acids, and the like. To determine the number of cells represented in a lysate, one can, e.g., obtain data from which to derive a standard function of ribosomal DNA (rDNA) versus numbers of cells, and interpolate the number of cells represented in an unknown lysate (e.g., a test sample) based on the amount of the rDNA present in the unknown lysate. A standard function can be an equation expressing the relationship between one quantity and another, such as, e.g., an assay input and assay output, or a constant proportional relationship between a number of cells and an amount of nucleic acid in a lysate of the cells. Typical standard functions can include, e.g., a standard curve plotting X-Y coordinates of related values on a chart, an equation established by regression analysis of standard assay results, or a constant ratio or proportion between related parameters. An expression of a standard function can be a “best fit” line on a paper chart, a ratio or line slope representing a proportionality between the cell numbers and their rDNA, an equation determined by regression analysis techniques, a result provided by a computer using an appropriate program, and the like, as is known in the art. For example, the number of cells represented in a test sample lysate can be determined by: obtaining a reference lysate from a known number of cells, quantitating the amount of genes encoding a ribosomal RNA in the reference lysate, determining a ratio of cell numbers to an amount of the rDNA in a sample, quantitating the amount of the rDNA in the test sample, and calculating the number of cells represented in the test sample lysate based on the ratio. The number of cells in a reference sample can be determined, e.g., by counting them using methods known in the art. For example, reference cells grown in suspension can be counted in a hemocytometer, in a Coulter counter, by a cell sorter, inferred by packed cell volume, and the like. Cells in a reference tissue can be counted microscopically, inferred from tissue volume, or counted as for suspended cells above after release by mechanical, chemical and/or enzymatic techniques. The reference cells can be normal cells, primary culture cells, cell lines, cells released from tissues, cells from biological fluids, and/or the like. The reference cells can be the same type as the test cells, or not. The cells can be uniformly the same or a mixture of different cell types. Additional information on standard functions, determination of cell number, and the like can be found in U.S. patent application publication 20080050746 entitled “Nucleic acid quantitation from tissue slides” by McMaster et al.

The reference nucleic acid can be quantitated in the reference and test samples by essentially any method with sufficient sensitivity and accuracy to provide a useful output. Preferably, reference nucleic acid determinations for both test and reference samples use the same methodology, to avoid interassay variables, but the methods need not be the same. Quantitation of the reference nucleic acid can be by, e.g., bDNA analysis, quantitative PCR, Northern blot analysis, in situ hybridizations, and the like. Quantitation of the reference nucleic acid is optionally multiplexed with analysis of the repeated sequence element. For example, in embodiments in which different nucleic acid targets are captured to different sets of particles or different positions on a solid support, the reference nucleic acid can be captured to yet another different set or position (e.g., with its own set of complementary capture extenders and a unique capture probe) and analyzed using its own complementary label extender(s).

The test sample in which copy number of the repeated sequence element is to be determined can be essentially any sample, e.g., containing or suspected of containing one or more nucleic acids desirably assayed for repeated sequence element(s). For example, the sample can be derived from a eukaryote, a vertebrate, an animal, a human, a plant, an insect, a protist, a fungus, a yeast, a cultured cell, a virus, a bacterium, a pathogen, and/or a microorganism. The sample optionally includes a cell lysate, an intercellular fluid, a bodily fluid (including, but not limited to, blood, serum, saliva, urine, sputum, or spinal fluid), and/or a conditioned culture medium, and is optionally derived from a tissue (e.g., a tissue homogenate), a biopsy, and/or a tumor.

The methods are optionally used for diagnosis or prognosis of a disease or other condition (e.g., cancer, diabetes, Huntington's disease, etc., e.g., by detecting telomere length or STR or VNTR copy number), monitoring response to treatment of a disease, screening for drug candidates (e.g., telomerase inhibitors), estimating age or identifying markers in forensics, and many other applications.

### Compositions, Kits, and Systems

Compositions, kits, and systems related to the methods are also features of the invention. Thus, one general class of embodiments provides a composition that includes a first set of one or more capture extenders, which first set of capture extenders is capable of hybridizing to a first nucleic acid target molecule that comprises multiple tandem copies of a repeated sequence element; a label extender (e.g., in multiple copies), which label extender is capable of hybridizing to at least one copy of the repeated sequence element or to a subsequence thereof; and a label probe system comprising a label, wherein a component of the label probe system is capable of hybridizing to the label extender. The composition optionally includes the first nucleic acid target molecule (e.g., in a test sample).

Essentially all of the features noted for the methods above apply to these embodiments as well, as relevant; for example, with respect to composition of the label probe system (e.g., inclusion of preamplifier, amplification multimer, and/or label probe), type of label, type, length, and/or copy number of the repeated sequence element, source of the nucleic acid and/or test sample, configuration of the label extender (e.g., to bind to a subsequence of the repeated sequence element, to one entire copy of the element, or to two or more tandem copies of the element), inclusion of blocking probes, a second (third, fourth, etc.) set of capture extenders for a second (third, fourth, etc.) nucleic acid target molecule, the second (third, fourth, etc.) target nucleic acid molecule, a solid support (including, e.g., a spatially addressable support or population of sets of identifiable particles), capture probe(s) (e.g., a single capture probe on a solid support, or an array of capture probes on a spatially addressable solid support or on distinguishable sets of particles), a reference nucleic acid, a set of one or more capture extenders capable of hybridizing to the reference nucleic acid, and/or at least one label extender capable of hybridizing to the reference nucleic acid, and/or the like.

Yet another general class of embodiments provides a kit for determining copy number of a repeated sequence element present in multiple tandem copies on a first nucleic acid target molecule. The kit includes a first set of one or more capture extenders, which first set of capture extenders is capable of hybridizing to the first nucleic acid target molecule; a label extender (e.g., multiple copies of the LE), which label extender is capable of hybridizing to at least one copy of the repeated sequence element or to a subsequence thereof; and a label probe system comprising a label, wherein a component of the label probe system is capable of hybridizing to the label extender; packaged in one or more containers.

The kit optionally also includes instructions for using the kit, e.g., to determine copy number of or length occupied by the repeated sequence element, one or more buffered solutions (e.g., lysis buffer, diluent, hybridization buffer, and/or wash buffer), one or more standards comprising one or more nucleic acids at known concentration (e.g., a reference nucleic acid or a nucleic acid including a known number of copies of the repeated sequence element), a second (third, fourth, etc.) set of one or more capture extenders for a second (third, fourth, etc.) nucleic acid target molecule, blocking probes, a solid support (e.g., a spatially addressable support or population of sets of identifiable particles), capture probe(s) (e.g., a single capture probe on a solid support, or an array of capture probes on a spatially addressable solid support or on distinguishable sets of particles), a set of one or more capture extenders capable of hybridizing to a reference nucleic acid, and/or at least one label extender capable of hybridizing to the reference nucleic acid.

Essentially all of the features noted for the methods above apply to these embodiments as well, as relevant; for example, with respect to composition of the label probe system (e.g., inclusion of preamplifier, amplification multimer, and/or label probe), type of label, type, length, and/or copy number of the repeated sequence element, source of the nucleic acid and/or test sample, configuration of the label extender (e.g., to bind to a subsequence of the repeated sequence element, to one entire copy of the element, or to two or more tandem copies of the element), and/or the like.

In one aspect, the invention includes systems, e.g., systems used to practice the methods herein and/or comprising the compositions described herein. The system can include, e.g., a fluid and/or microsphere handling element, a fluid and/or microsphere containing element, a laser for exciting a fluorescent label and/or fluorescent microspheres, a detector for detecting light emissions from a chemiluminescent reaction or fluorescent emissions from a fluorescent label and/or fluorescent microspheres, and/or a robotic element that moves other components of the system from place to place as needed (e.g., a multiwell plate handling element). For example, in one class of embodiments, a composition of the invention is contained in a flow cytometer, a Luminex 100™ or HTS™ instrument, a microplate reader, a microarray reader, a luminometer, a colorimeter, or like instrument.

The system can optionally include a computer. The computer can include appropriate software for receiving user instructions, either in the form of user input into a set of parameter fields, e.g., in a GUI, or in the form of preprogrammed instructions, e.g., preprogrammed for a variety of different specific operations. The software optionally converts these instructions to appropriate language for controlling the operation of components of the system (e.g., for controlling a fluid handling element, robotic element and/or laser). The computer can also receive data from other components of the system, e.g., from a detector, and can interpret the data, provide it to a user in a human readable format, or use that data to initiate further operations, in accordance with any programming by the user.

### Labels

A wide variety of labels are well known in the art and can be adapted to the practice of the present invention. For example, luminescent labels and light-scattering labels (e.g., colloidal gold particles) have been described. See, e.g., Csaki et al. (2002) “Gold nanoparticles as novel label for DNA diagnostics” Expert Rev Mol Diagn 2:187-93.

As another example, a number of fluorescent labels are well known in the art, including but not limited to, hydrophobic fluorophores (e.g., phycoerythrin, rhodamine, Alexa Fluor 488 and fluorescein), green fluorescent protein (GFP) and variants thereof (e.g., cyan fluorescent protein and yellow fluorescent protein), and quantum dots. See, e.g., Haughland (2003) Handbook of Fluorescent Probes and Research Products, Ninth Edition or Web Edition, from Molecular Probes, Inc., or The Handbook: A Guide to Fluorescent Probes and Labeling Technologies, Tenth Edition or Web Edition (2009) from Invitrogen (available on the world wide web at probes (dot) invitrogen (dot) com/handbook) for descriptions of fluorophores emitting at various different wavelengths (including tandem conjugates of fluorophores that can facilitate simultaneous excitation and detection of multiple labeled species). For use of quantum dots as labels for biomolecules, see, e.g., Dubertret et al. (2002) Science 298:1759; Nature Biotechnology (2003) 21:41-46; and Nature Biotechnology (2003) 21:47-51.

Labels can be introduced to molecules, e.g. polynucleotides, during synthesis or by postsynthetic reactions by techniques established in the art; for example, kits for fluorescently labeling polynucleotides with various fluorophores are available from Molecular Probes, Inc. (www (dot) molecularprobes (dot) com), and fluorophore-containing phosphoramidites for use in nucleic acid synthesis are commercially available. Similarly, signals from the labels (e.g., absorption by and/or fluorescent emission from a fluorescent label) can be detected by essentially any method known in the art. For example, multicolor detection, detection of FRET, fluorescence polarization, and the like are well known in the art.

### Microspheres

Microspheres are preferred particles in certain embodiments described herein since they are generally stable, are widely available in a range of materials, surface chemistries and uniform sizes, and can be fluorescently dyed. Microspheres can be distinguished from each other by identifying characteristics such as their size (diameter) and/or their fluorescent emission spectra, for example.

Luminex Corporation (www (dot) luminexcorp (dot) com), for example, offers 100 sets of uniform diameter polystyrene microspheres. The microspheres of each set are internally labeled with a distinct ratio of two fluorophores. A flow cytometer or other suitable instrument can thus be used to classify each individual microsphere according to its predefined fluorescent emission ratio. Fluorescently-coded microsphere sets are also available from a number of other suppliers, including Radix Biosolutions (www (dot) radixbiosolutions (dot) com) and Upstate Biotechnology (www (dot) upstatebiotech (dot) com). Alternatively, BD Biosciences (www (dot) bd (dot) com) and Bangs Laboratories, Inc. (www (dot) bangslabs (dot) com) offer microsphere sets distinguishable by a combination of fluorescence and size. As another example, microspheres can be distinguished on the basis of size alone, but fewer sets of such microspheres can be multiplexed in an assay because aggregates of smaller microspheres can be difficult to distinguish from larger microspheres.

Microspheres with a variety of surface chemistries are commercially available, from the above suppliers and others (e.g., see additional suppliers listed in Kellar and Iannone (2002) “Multiplexed microsphere-based flow cytometric assays” Experimental Hematology 30:1227-1237 and Fitzgerald (2001) “Assays by the score” The Scientist 15[11]:25). For example, microspheres with carboxyl, hydrazide or maleimide groups are available and permit covalent coupling of molecules (e.g., polynucleotide capture probes with free amine, carboxyl, aldehyde, sulfhydryl or other reactive groups) to the microspheres. As another example, microspheres with surface avidin or streptavidin are available and can bind biotinylated capture probes; similarly, microspheres coated with biotin are available for binding capture probes conjugated to avidin or streptavidin. In addition, services that couple a capture reagent of the customer's choice to microspheres are commercially available, e.g., from Radix Biosolutions (www (dot) radixbiosolutions (dot) com).

Protocols for using such commercially available microspheres (e.g., methods of covalently coupling polynucleotides to carboxylated microspheres for use as capture probes, methods of blocking reactive sites on the microsphere surface that are not occupied by the polynucleotides, methods of binding biotinylated polynucleotides to avidin-functionalized microspheres, and the like) are typically supplied with the microspheres and are readily utilized and/or adapted by one of skill. In addition, coupling of reagents to microspheres is well described in the literature. For example, see Yang et al. (2001) “BADGE, Beads Array for the Detection of Gene Expression, a high-throughput diagnostic bioassay” Genome Res. 11:1888-98; Fulton et al. (1997) “Advanced multiplexed analysis with the FlowMetrix™ system” Clinical Chemistry 43:1749-1756; Jones et al. (2002) “Multiplex assay for detection of strain-specific antibodies against the two variable regions of the G protein of respiratory syncytial virus” 9:633-638; Camilla et al. (2001) “Flow cytometric microsphere-based immunoassay: Analysis of secreted cytokines in whole-blood samples from asthmatics” Clinical and Diagnostic Laboratory Immunology 8:776-784; Martins (2002) “Development of internal controls for the Luminex instrument as part of a multiplexed seven-analyte viral respiratory antibody profile” Clinical and Diagnostic Laboratory Immunology 9:41-45; Kellar and Iannone (2002) “Multiplexed microsphere-based flow cytometric assays” Experimental Hematology 30:1227-1237; Oliver et al. (1998) “Multiplexed analysis of human cytokines by use of the FlowMetrix system” Clinical Chemistry 44:2057-2060; Gordon and McDade (1997) “Multiplexed quantification of human IgG, IgA, and IgM with the FlowMetrix™ system” Clinical Chemistry 43:1799-1801; U.S. Pat. No. 5,981,180 entitled “Multiplexed analysis of clinical specimens apparatus and methods” to Chandler et al. (Nov. 9, 1999); U.S. Pat. No. 6,449,562 entitled “Multiplexed analysis of clinical specimens apparatus and methods” to Chandler et al. (Sep. 10, 2002); and references therein.

Methods of analyzing microsphere populations (e.g. methods of identifying microsphere subsets by their size and/or fluorescence characteristics, methods of using size to distinguish microsphere aggregates from single uniformly sized microspheres and eliminate aggregates from the analysis, methods of detecting the presence or absence of a fluorescent label on the microsphere subset, and the like) are also well described in the literature. See, e.g., the above references.

Suitable instruments, software, and the like for analyzing microsphere populations to distinguish subsets of microspheres and to detect signal from a label (e.g., a fluorescently labeled label probe) on each subset are commercially available. For example, flow cytometers are widely available, e.g., from Becton-Dickinson (www (dot) bd (dot) com) and Beckman Coulter (www (dot) beckman (dot) com). Luminex 100™ and Luminex HTS™ systems (which use microfluidics to align the microspheres and two lasers to excite the microspheres and the label) are available from Luminex Corporation (www (dot) luminexcorp (dot) com); the similar Bio-Plex™ Protein Array System is available from Bio-Rad Laboratories, Inc. (www (dot) bio-rad (dot) com). A confocal microplate reader suitable for microsphere analysis, the FMAT™ System 8100, is available from Applied Biosystems (www (dot) appliedbiosystems (dot) com).

As another example of particles that can be adapted for use in the present invention, sets of microbeads that include optical barcodes are available from CyVera Corporation (www (dot) cyvera (dot) com). The optical barcodes are holographically inscribed digital codes that diffract a laser beam incident on the particles, producing an optical signature unique for each set of microbeads.

### Arrays

In an array of capture probes on a solid support (e.g., a membrane, a glass or plastic slide, a silicon or quartz chip, a plate, or other spatially addressable solid support), each capture probe is typically bound (e.g., electrostatically or covalently bound, directly or via a linker) to the support at a unique selected location. Methods of making, using, and analyzing such arrays (e.g., microarrays) are well known in the art. See, e.g., Baldi et al. (2002) DNA Microarrays and Gene Expression: From Experiments to Data Analysis and Modeling, Cambridge University Press; Beaucage (2001) “Strategies in the preparation of DNA oligonucleotide arrays for diagnostic applications” Curr Med Chem 8:1213-1244; Schena, ed. (2000) Microarray Biochip Technology, pp. 19-38, Eaton Publishing; and references therein. Arrays of pre-synthesized polynucleotides can be formed (e.g., printed), for example, using commercially available instruments such as a GMS 417 Arrayer (Affymetrix, Santa Clara, Calif.). Alternatively, the polynucleotides can be synthesized at the selected positions on the solid support; see, e.g., U.S. Pat. No. 6,852,490 and U.S. Pat. No. 6,306,643, each to Gentanlen and Chee entitled “Methods of using an array of pooled probes in genetic analysis.”

Suitable solid supports are commercially readily available. For example, a variety of membranes (e.g., nylon, PVDF, and nitrocellulose membranes) are commercially available, e.g., from Sigma-Aldrich, Inc. (www (dot) sigmaaldrich (dot) com). As another example, surface-modified and pre-coated slides with a variety of surface chemistries are commercially available, e.g., from TeleChem International (www (dot) arrayit (dot) com), Corning, Inc. (Corning, N.Y.), or Greiner Bio-One, Inc. (www (dot) greinerbiooneinc (dot) com). For example, silanated and silyated slides with free amino and aldehyde groups, respectively, are available and permit covalent coupling of molecules (e.g., polynucleotides with free aldehyde, amine, or other reactive groups) to the slides. As another example, slides with surface streptavidin are available and can bind biotinylated capture probes. In addition, services that produce arrays of polynucleotides of the customer's choice are commercially available, e.g., from TeleChem International (www (dot) arrayit (dot) com) and Agilent Technologies (Palo Alto, Calif.).

Suitable instruments, software, and the like for analyzing arrays to distinguish selected positions on the solid support and to detect the presence or absence of a label (e.g., a fluorescently labeled label probe) at each position are commercially available. For example, microarray readers are available, e.g., from Agilent Technologies (Palo Alto, Calif.), Affymetrix (Santa Clara, Calif.), and Zeptosens (Switzerland).

### Molecular Biological Techniques

In practicing the present invention, many conventional techniques in molecular biology, microbiology, and recombinant DNA technology are optionally used. These techniques are well known and are explained in, for example, Berger and Kimmel, Guide to Molecular Cloning Techniques, Methods in Enzymology volume 152 Academic Press, Inc., San Diego, Calif.; Sambrook et al., Molecular Cloning—A Laboratory Manual (3rd Ed.), Vol. 1-3, Cold Spring Harbor Laboratory, Cold Spring Harbor, N.Y., 2000 and Current Protocols in Molecular Biology, F. M. Ausubel et al., eds., Current Protocols, a joint venture between Greene Publishing Associates, Inc. and John Wiley & Sons, Inc., (supplemented through 2009). Other useful references, e.g. for cell isolation and culture (e.g., for subsequent nucleic acid or protein isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (Eds.) (1995) Plant Cell, Tissue and Organ Culture; Fundamental Methods Springer Lab Manual, Springer-Verlag (Berlin Heidelberg New York) and Atlas and Parks (Eds.) The Handbook of Microbiological Media (1993) CRC Press, Boca Raton, Fla.

Making Polynucleotides

Methods of making nucleic acids (e.g., by in vitro amplification, purification from cells, or chemical synthesis), methods for manipulating nucleic acids (e.g., by restriction enzyme digestion, ligation, etc.) and various vectors, cell lines and the like useful in manipulating and making nucleic acids are described in the above references. In addition, methods of making branched polynucleotides (e.g., amplification multimers) are described in U.S. Pat. No. 5,635,352, U.S. Pat. No. 5,124,246, U.S. Pat. No. 5,710,264, and U.S. Pat. No. 5,849,481, as well as in other references mentioned above.

In addition, essentially any polynucleotide (including, e.g., labeled or biotinylated polynucleotides) can be custom or standard ordered from any of a variety of commercial sources, such as The Midland Certified Reagent Company (www (dot) mcrc (dot) com), The Great American Gene Company (www (dot) genco (dot) com), ExpressGen Inc. (www (dot) expressgen (dot) com), Qiagen (oligos (dot) qiagen (dot) com) and many others.

A label, biotin, or other moiety can optionally be introduced to a polynucleotide, either during or after synthesis. For example, a biotin phosphoramidite can be incorporated during chemical synthesis of a polynucleotide. Alternatively, any nucleic acid can be biotinylated using techniques known in the art; suitable reagents are commercially available, e.g., from Pierce Biotechnology (www (dot) piercenet (dot) com). Similarly, any nucleic acid can be fluorescently labeled, for example, by using commercially available kits such as those from Molecular Probes, Inc. (www (dot) molecularprobes (dot) com) or Pierce Biotechnology (www (dot) piercenet (dot) com) or by incorporating a fluorescently labeled phosphoramidite during chemical synthesis of a polynucleotide.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims. Accordingly, the following examples are offered to illustrate, but not to limit, the claimed invention.

### Example 1

### Measurement of Mean Telomere Length

The following sets forth a series of experiments that demonstrate measurement of average telomere length using a bDNA assay with a single capture extender and a single label extender complementary to the human telomeric repeat sequence.

The sequence of the label extender was ccctaaccctaaccctaaccctaaTTTTTagtcaaagcatgaagttaccgtttt (SEQ ID NO:4) and that of the capture extender was ccctaaccctaaccctaaccctaaTTTTTctcttggaaagaaagt (SEQ ID NO:5); the underlined region is complementary to four tandem copies of the telomeric repeat sequence. Other reagents (bDNA amplifier, alkaline phosphatase-conjugated label probe, lysis and wash buffers, etc.) were from the commercially available QuantiGene® 2.0 Reagent System (Affymetrix, Inc.; www dot panomics dot com).

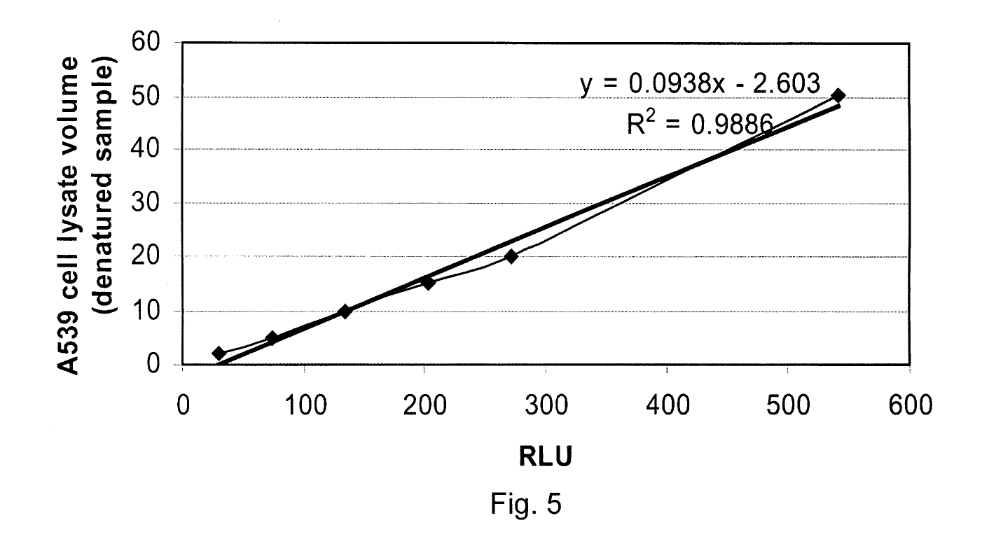

Lysate was prepared from A539 cells and a bDNA assay was performed basically as described in the instructions accompanying the QuantiGene® 2.0 reagents (for example, concentrations of the capture extender and label extender were 25 nM and 100 nM, respectively, in the target-probe hybridization mixture), except that all hybridizations were performed at 47° C. The resulting luminescent signal measured for different volumes of cell lysate is shown in Table 1 and FIG. 5.

TABLE 1
Results of bDNA assay for telomere length using denatured samples

| | mean^{a} | minus bk^{b} | CV^{c} | S/N^{d} |
|---|---|---|---|---|
| CE + LE | 11.88 | | | |
| CE + LE + 50 μL | 553.82 | 541.94 | 0.91 | 46.63 |
| CE + LE + 20 μL | 283.62 | 271.74 | 6.59 | 23.88 |
| CE + LE + 15 μL | 214.73 | 202.85 | 2.10 | 18.08 |
| CE + LE + 10 μL | 145.76 | 133.89 | 2.98 | 12.27 |
| CE + LE + 5 μL | 86.43 | 74.55 | 5.14 | 7.28 |
| CE + LE + 2 μL | 41.10 | 29.23 | 2.79 | 3.46 |

^{a}of three replicates
^{b}background subtracted signal
^{c}standard deviation/mean
^{d}signal to background ratio

The results demonstrate that, using a single capture extender and label extender having the same target recognition sequence, average telomere length can be measured. Optionally, total DNA is determined, e.g., using a bDNA assay with a probe set for 18sDNA or another reference nucleic acid. Telomere length for a given sample can then be defined as the signal for the telomere probe set divided by the signal for the 18sDNA (or other reference) probe set, and compared to a reference or control sample, for example, to compare different cell types, sources, etc. to determine if the average telomere length in the sample is longer, shorter, or comparable to that in the control.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1-43. (canceled)

44. A method of diagnosing or determining prognosis of cancer by detecting copy number of a repeated sequence element, the method comprising:
providing a test sample comprising at least one nucleic acid target molecule comprising copies of the repeated sequence element;
providing multiple copies of a label extender, which label extender is capable of hybridizing to at least one copy of the repeated sequence element or to a subsequence thereof;
providing a label probe system comprising a label, wherein a component of the label probe system is capable of hybridizing to the label extender;
hybridizing the label extender copies to the copies of the repeated sequence element or subsequence thereof on the at least one nucleic acid target molecule;
hybridizing the label probe system to the label extender copies;
detecting a signal from the label; and
correlating an intensity of the signal with a number of copies of the repeated sequence element.

45. The method of claim 44, wherein the hybridizing steps are performed with the at least one nucleic acid target molecule inside a cell.

46. The method of claim 44, comprising capturing the at least one nucleic acid target molecule on a solid support prior to detecting the signal from the label.

47. The method of claim 46, wherein capturing the at least one nucleic acid target molecule on the solid support comprises:
providing a first set of one or more capture extenders, which first set of capture extenders is capable of hybridizing to the at least one nucleic acid target molecule;
hybridizing the first set of capture extenders to the at least one nucleic acid target molecule; and
associating the first set of capture extenders with the solid support.

48. The method of claim 47, wherein a first capture probe is bound to the solid support, and wherein associating the first set of capture extenders with the solid support comprises hybridizing the capture extenders to the first capture probe.

49. The method of claim 44, wherein the label probe system comprises a preamplifier, a plurality of amplification multimers, and a multiplicity of label probes, wherein the preamplifier is capable of hybridizing simultaneously to the label extender and to the plurality of amplification multimers, and wherein the amplification multimer is capable of hybridizing simultaneously to the preamplifier and to a plurality of the label probes.

50. The method of claim 49, wherein the label probe comprises the label.

51. The method of claim 44, wherein the at least one nucleic acid target molecule comprises at least one RNA molecule.

52. The method of claim 44, wherein the repeated sequence element is 50 nucleotides or less in length.

53. The method of claim 44, wherein the repeated sequence element is 25 nucleotides or less in length.

54. The method of claim 44, wherein the repeated sequence element is a satellite repeat.

55. The method of claim 44, wherein the label extender is capable of hybridizing to one copy of the repeated sequence element.

56. The method of claim 55, wherein the label extender is capable of hybridizing to a subsequence of the repeated sequence element.

57. The method of claim 44, wherein correlating the intensity of the signal with the number of copies of the repeated sequence element comprises:
providing a standard function for cell number or amount of cellular nucleic acid input versus quantity of a reference nucleic acid;
quantitating the reference nucleic acid from the test sample;
determining a cell number or amount of cellular nucleic acid for the test sample based on the standard function and the quantity of the reference nucleic acid in the test sample; and
normalizing the intensity of the signal and/or the number of copies to the cell number or amount of cellular nucleic acid.

58. The method of claim 57, wherein the reference nucleic acid is selected from the group consisting of a ribosomal DNA, an Alu sequence, and a β-globin gene.

59. The method of claim 44, wherein correlating the intensity of the signal with the number of copies of the repeated sequence element comprises normalizing the intensity to an intensity measured for a reference nucleic acid.

60. The method of claim 59, wherein the reference nucleic acid is selected from the group consisting of a ribosomal DNA, an Alu sequence, and a β-globin gene.

61. The method of claim 44, wherein correlating the intensity of the signal with the number of copies of the repeated sequence element comprises comparing the intensity to an intensity for a control or reference sample and expressing the copy number qualitatively relative to the control or reference sample.

62. The method of claim 44, further comprising:
providing multiple copies of a second label extender, which second label extender is capable of hybridizing to at least one copy of a second repeated sequence element or to a subsequence thereof, which second repeated sequence element is present on the at least one nucleic acid target molecule and/or on at least one other nucleic acid molecule in the test sample;
providing a second label probe system comprising a different, second label, wherein a component of the second label probe system is capable of hybridizing to the second label extender;
hybridizing the second label extender copies to the copies of the second repeated sequence element or subsequence thereof; and
hybridizing the second label probe system to the second label extender copies.

63. The method of claim 44, wherein the test sample is derived from a tissue, a biopsy, and/or a tumor.

64. A method of detecting copy number of a repeated sequence element, the method comprising:
providing a test sample comprising at least one nucleic acid target molecule comprising copies of the repeated sequence element;
providing multiple copies of a label extender, which label extender is capable of hybridizing to at least one copy of the repeated sequence element or to a subsequence thereof;
providing a label probe system comprising a label, wherein a component of the label probe system is capable of hybridizing to the label extender;
hybridizing the label extender copies to the copies of the repeated sequence element or subsequence thereof on the at least one nucleic acid target molecule;
hybridizing the label probe system to the label extender copies;
detecting a signal from the label; and
correlating an intensity of the signal with a number of copies of the repeated sequence element.
